(19) United States
(12) Patent Application Publication
(10) Pub. No.: US 2005/0137520 A1
(43) Pub. Date: Jun. 23, 2005

(54) Catheter with ultrasound-controllable porous membrane

(76) Inventors: Peter R. Rule, Los Altos, CA (US); Douglas R. Hansmann, Bainbridge Island, WA (US); Robert L. Wilcox, Bothell, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/977,502

(22) Filed: Oct. 29, 2004

Related U.S. Application Data
(60) Provisional application No. 60/515,263, filed on Oct. 29, 2003.

(51) Int. Cl. A61B 17/20

(52) U.S. Cl. 604/22

(57) ABSTRACT

A catheter system for delivering ultrasonic energy and a therapeutic compound to a treatment site within a patient's vasculature comprises a tubular body having an energy delivery section. The catheter system further comprises a fluid delivery lumen extending at least partially through the tubular body. The catheter system further comprises a semi-permeable membrane positioned along a portion of the fluid delivery lumen. The membrane has an increased porosity when exposed to ultrasonic energy. The catheter system further comprises an inner core configured for insertion into the tubular body. The inner core comprises an elongate electrical conductor having a plurality of flattened regions. Each flattened region has a first flat side and a second flat side opposite the first flat side. The inner core further comprises a plurality of ultrasound radiating members mounted in pairs to the flattened regions of the elongate electrical conductor. A first ultrasound radiating member is mounted to the first flat side of the elongate electrical conductor, and a second ultrasound radiating member is mounted to the second flat side of the elongate electrical conductor. The inner core further comprises wiring such that a voltage can be applied from the elongate electrical conductor across the first and second ultrasound radiating members allowing the first and second ultrasound radiating members to be driven simultaneously.

### PRIORITY APPLICATION

This application claims the benefit of U.S. Provisional Application 60/515,263, filed 29 Oct. 2003, the entire disclosure of which is hereby incorporated by reference herein.

### FIELD OF THE INVENTION

The present invention relates generally to medical devices and procedures, and more specifically to ultrasound catheter systems capable of controlling the delivery of a therapeutic compound using ultrasonic energy.

### BACKGROUND OF THE INVENTION

Human blood vessels occasionally become occluded by clots, plaque, thrombi, emboli or other substances that reduce the blood carrying capacity of the vessel. Cells that rely on blood passing through the occluded vessel for nourishment may die if the vessel remains occluded. This often results in grave consequences for a patient, particularly in the case of cells such as brain cells or heart cells.

Accordingly, several techniques are being developed for removing an occlusion from a blood vessel. Examples of such techniques include the introduction into the vasculature of therapeutic compounds—including enzymes—that dissolve blood clots. When such therapeutic compounds are introduced into the bloodstream, often systematic effects result, rather than local effects. Accordingly, recently catheters have been used to introduce therapeutic compounds at or near the occlusion. Mechanical techniques have also been used to remove an occlusion from a blood vessel. For example, ultrasonic catheters have been developed that include an ultrasound radiating member that is positioned in or near the occlusion. Ultrasonic energy is then used to ablate the occlusion. Other techniques involve the use of lasers and mechanical thrombectomy and/or clot macerator devices.

One particularly effective apparatus and method for removing an occlusion uses the combination of ultrasonic energy and a therapeutic compounds that removes an occlusion. Using such systems, a blockage is removed by advancing an ultrasound catheter through the patient's vasculature to deliver therapeutic compounds containing dissolution compounds directly to the blockage site. To enhance the therapeutic effects of the therapeutic compound, ultrasonic energy is emitted into the dissolution compound and/or the surrounding tissue. See, for example, U.S. Pat. No. 6,001,069.

### SUMMARY OF THE INVENTION

An improved ultrasonic catheter has been developed. In certain embodiments, this catheter is capable of delivering a specific quantity of therapeutic compound to a selected treatment location within a patient's vasculature. In such embodiments, control over location and quantity of therapeutic compound delivery is accomplished through the use of a membrane having a variable porosity that changes when exposed to ultrasonic energy. Accurate delivery of therapeutic compound, both in location and quantity, can advantageously reduce patient complications and enhance treatment efficacy.

In one embodiment of the present invention, a catheter system for delivering ultrasonic energy and a therapeutic compound to a treatment site within a patient's vasculature comprises a tubular body having an energy delivery section. The catheter system further comprises a fluid delivery lumen extending at least partially through the tubular body. The catheter system further comprises a semi-permeable membrane positioned along a portion of the fluid delivery lumen. The membrane has an increased porosity when exposed to ultrasonic energy. The catheter system further comprises an inner core configured for insertion into the tubular body. The inner core comprises an elongate electrical conductor having a plurality of flattened regions. Each flattened region has a first flat side and a second flat side opposite the first flat side. The inner core further comprises a plurality of ultrasound radiating members mounted in pairs to the flattened regions of the elongate electrical conductor. A first ultrasound radiating member is mounted to the first flat side of the elongate electrical conductor, and a second ultrasound radiating member is mounted to the second flat side of the elongate electrical conductor. The inner core further comprises wiring such that a voltage can be applied from the elongate electrical conductor across the first and second ultrasound radiating members allowing the first and second ultrasound radiating members to be driven simultaneously.

In another embodiment of the present invention, a catheter comprises an elongate outer sheath with an exterior surface. A distal end portion of the outer sheath has a diameter of less than about 5 French. The outer sheath defines a central lumen extending longitudinally therethrough. the catheter further comprises an elongate inner core extending through the central lumen of the outer sheath and ending at an exit port located at a catheter distal tip. The inner core defines a delivery lumen adapted for delivery of a therapeutic compound through the delivery lumen an out the exit port to a treatment site. The catheter further comprises a cylindrical ultrasound radiating member coupled along the distal end portion of the inner core and located distal to the outer sheath. The catheter further comprises a semi-permeable membrane covering the exit port. A fluid passing from the delivery lumen to the treatment site crosses the semi-permeable membrane.

In another embodiment of the present invention, a catheter configured to be positioned within a patient's vasculature comprises a fluid delivery lumen. The catheter further comprises an ultrasound radiating member positioned adjacent to at least a portion of the fluid delivery lumen. The catheter further comprises a semi-permeable sheath covering at least a portion of the fluid delivery lumen. A fluid passing from the fluid delivery lumen to the patient's vasculature crosses the sheath. The sheath has an increased porosity when exposed to ultrasonic energy.

In another embodiment of the present invention, a method comprises positioning a catheter at a treatment site within a patient's vasculature. The catheter includes an ultrasound radiating member and a fluid delivery lumen. An obstruction is located at the treatment site. The method further comprises passing a therapeutic compound through the fluid delivery lumen. The method further comprises passing a control signal to the ultrasound radiating member. Ultrasonic energy is generated at the treatment site, and generation of ultrasonic energy causes at least a portion of the therapeutic compound to pass from the fluid delivery lumen, through a semi-permeable membrane, and to the patient's vasculature.

### BRIEF DESCRIPTION OF THE DRAWINGS

Exemplary embodiments of the ultrasonic catheter disclosed herein are illustrated in the accompanying drawings, which are for illustrative purposes only. The drawings comprise the following figures, in which like numerals indicate like parts.

FIG. 1 is a schematic illustration of an ultrasonic catheter configured for insertion into large vessels of the human body.

FIG. 2 is a cross-sectional view of the ultrasonic catheter of FIG. 1 taken along line 2-2.

FIG. 3 is a schematic illustration of an elongate inner core configured to be positioned within the central lumen of the catheter illustrated in FIG. 2.

FIG. 4 is a cross-sectional view of the elongate inner core of FIG. 3 taken along line 4-4.

FIG. 5 is a schematic wiring diagram illustrating an exemplary technique for electrically connecting five groups of ultrasound radiating members to form an ultrasound assembly.

FIG. 6 is a schematic wiring diagram illustrating an exemplary technique for electrically connecting one of the groups of FIG. 5.

FIG. 7A is a schematic illustration of the ultrasound assembly of FIG. 5 housed within the inner core of FIG. 4.

FIG. 7B is a cross-sectional view of the ultrasound assembly of FIG. 7A taken along line 7B-7B.

FIG. 7C is a cross-sectional view of the ultrasound assembly of FIG. 7A taken along line 7C-7C.

FIG. 7D is a side view of an ultrasound assembly center wire twisted into a helical configuration.

FIG. 8 illustrates the energy delivery section of the inner core of FIG. 4 positioned within the energy delivery section of the tubular body of FIG. 2.

FIG. 9 illustrates a wiring diagram for connecting a plurality of temperature sensors with a common wire.

FIG. 10 is a block diagram of a feedback control system for use with an ultrasonic catheter.

FIG. 11A is a side view of a treatment site.

FIG. 11B is a side view of the distal end of an ultrasonic catheter positioned at the treatment site of FIG. 11A.

FIG. 11C is a cross-sectional view of the distal end of the ultrasonic catheter of FIG. 11B positioned at the treatment site before a treatment.

FIG. 11D is a cross-sectional view of the distal end of the ultrasonic catheter of FIG. 11C, wherein an inner core has been inserted into the tubular body to perform a treatment.

FIG. 12 is a side view of an ultrasound catheter that is particularly well suited for insertion into small blood vessels of the human body.

FIG. 13A is a cross-sectional view of a distal end of the ultrasound catheter of FIG. 12.

FIG. 13B is a cross-sectional view of the ultrasound catheter of FIG. 12 taken through line 13B-13B of FIG. 13A.

FIG. 14A is a cross-sectional view of a distal end of an ultrasound catheter, which includes therapeutic compound delivery ports and a membrane with ultrasound-controllable porosity.

FIG. 14B is a cross-sectional view of the distal end of the ultrasound catheter of FIG. 14A.

FIG. 15A is a cross-sectional view of a distal end of an ultrasound catheter that includes a material with ultrasound-controllable porosity.

FIG. 15B is a cross-sectional view of the distal end of the ultrasound catheter of FIG. 15A.

FIG. 16 is a schematic diagram of an exemplary embodiment of an apparatus configured for laboratory monitoring of a horizontally-oriented membrane having ultrasound-controllable porosity.

FIG. 17A is a schematic diagram of an exemplary embodiment of an apparatus configured for laboratory monitoring of a vertically-oriented membrane having ultrasound-controllable porosity.

FIG. 17B is a schematic diagram of another exemplary embodiment of an apparatus configured for laboratory monitoring of a vertically-oriented membrane having ultrasound-controllable porosity.

FIG. 18 is a schematic diagram of driving electronics used to control an ultrasound radiating member.

### DETAILED DESCRIPTION OF PREFERRED EMBODIMENTS

As described above, ultrasonic catheters have been developed that are capable of controlling location and quantity of therapeutic compound delivery. Certain embodiments of such catheters use a membrane having a variable porosity that changes when exposed to ultrasonic energy. Exemplary embodiments of these ultrasonic catheters, including exemplary methods of use, are described herein.

The ultrasonic catheters described herein can be used to enhance the therapeutic effects of therapeutic compounds at a treatment site within a patient's body. As used herein, the term “therapeutic compound” refers broadly, without limitation, to a drug, medicament, dissolution compound, genetic material or any other substance capable of effecting physiological functions. Additionally, any mixture comprising any such substances is encompassed within this definition of “therapeutic compound”, as well as any substance falling within the ordinary meaning of these terms. The enhancement of the effects of therapeutic compounds using ultrasonic energy is described in U.S. Pat. Nos. 5,318,014, 5,362,309, 5,474,531, 5,628,728, 6,001,069, 6,096,000, 6,210,356 and 6,296,619. Specifically, for applications that treat human blood vessels that have become partially or completely occluded by plaque, thrombi, emboli or other substances that reduce the blood carrying capacity of a vessel, suitable therapeutic compounds include, but are not limited to, an aqueous solution containing heparin, urokinase, streptokinase, TPA and BB-10153 (manufactured by British Biotech, Oxford, UK).

Certain features and aspects of the ultrasonic catheters disclosed herein may also find utility in applications where the ultrasonic energy itself provides a therapeutic effect. Examples of such therapeutic effects include preventing or reducing stenosis and/or restenosis; tissue ablation, abrasion or disruption; promoting temporary or permanent physiological changes in intracellular or intercellular structures; and rupturing micro-balloons or micro-bubbles for therapeutic compound delivery. Further information about such methods can be found in U.S. Pat. Nos. 5,269,291 and 5,431,663. Further information about using cavitation to produce biological effects can be found in U.S. Pat. No. RE36,939. Additionally, the methods and devices disclosed herein can also be used in applications that do not require the use of a catheter. For example, the methods and devices disclosed herein can be used to enhance hyperthermic drug treatment or to cause transdermal enhancement of the therapeutic effects of drugs, medication, pharmacological agents, or other therapeutic compounds at a specific site within the body. Certain methods and devices disclosed herein can also be used to provide a therapeutic or diagnostic effect without the use of a therapeutic compound. See, for example, U.S. Pat. Nos. 4,821,740; 4,953,565; 5,007,438 and 6,096,000.

Certain embodiments described herein provide an ultrasound catheter that is well suited for use in the treatment of small blood vessels or other body lumens having a small inner diameter. Such embodiments can be used to enhance the therapeutic effects of drugs, medication, pharmacological agents and other therapeutic compounds at a treatment site within the body. See, for example, U.S. Pat. Nos. 5,318,014; 5,362,309; 5,474,531; 5,628,728; 6,001,069; and 6,210,356. Certain embodiments described herein are particularly well suited for use in the treatment of thrombotic occlusions in small blood vessels, such as, for example, the cerebral arteries. Additionally, certain embodiments described herein can be used in other therapeutic applications, such as, for example, performing gene therapy (see, for example, U.S. Pat. No. 6,135,976), and activating light activated drugs for producing targeted tissue death (see, for example, U.S. Pat. No. 6,176,842). Moreover, such therapeutic applications can be used in wide variety of locations within the body, such as, for example, in other parts of the circulatory system, in solid tissues, in duct systems and in body cavities. Certain of the ultrasound catheters disclosed herein, and variations thereof, can also be used in other medical applications, such as, for example, diagnostic and imaging applications.

For purposes of summarizing the invention and the advantages achieved over the prior art, certain objects and advantages of the invention have been described above. It is to be understood that not necessarily all such objects or advantages may be achieved in accordance with any particular embodiment of the invention. Thus, for example, the invention may be embodied or carried out in a manner that achieves or optimizes one advantage or group of advantages as taught herein without necessarily achieving other objects or advantages as may be taught or suggested herein.

The embodiments disclosed herein are intended to be within the scope of the present invention. These and other embodiments should be apparent based on the following detailed description, which refers to the attached figures. The present invention is not limited to any particular disclosed embodiment, but is limited only by the claims set forth herein.

Definitions.

As used herein, the terms “ultrasound energy” and “ultrasonic energy” are used broadly, and include their ordinary meanings, and further include mechanical energy transferred through pressure or compression waves with a frequency greater than about 20 kHz. In one embodiment, the waves of the ultrasonic energy have a frequency between about 500 kHz and about 20 MHz, and in another embodiment the waves of ultrasonic energy have a frequency between about 1 MHz and about 3 MHz. In yet another embodiment, the waves of ultrasonic energy have a frequency of about 3 MHz.

As used herein, the term “catheter” is used broadly, and includes its ordinary meaning, and further includes an elongate flexible tube configured to be inserted into the body of a patient, such as, for example, a body cavity, duct or vessel.

As used herein, the term “therapeutic compound” refers, in addition to its ordinary meaning, to a drug, medicament, dissolution compound, genetic material, or any other substance capable of effecting physiological functions. Additionally, a mixture comprising such substances is encompassed within this definition of “therapeutic compound”.

As used herein, the term “end” refers, in addition to its ordinary meaning, to a region, such that “proximal end” includes “proximal region”, and “distal end” includes “distal region”.

As used herein, the term “proximal element joint” refers generally, and in addition to its ordinary meaning, to a region where a proximal portion of an ultrasound radiating member is attached to other components of an ultrasound catheter.

As used herein, the term “treatment site” refers generally, and in addition to its ordinary meaning, to a region where a medical procedure is performed within a patient's body. Where the medical procedure is a treatment configured to reduce an occlusion within the patient's vasculature, the term “treatment site” refers to the region of the obstruction, as well as the region upstream of the obstruction and the region downstream of the obstruction.

Overview of a Large Vessel Ultrasound Catheter.

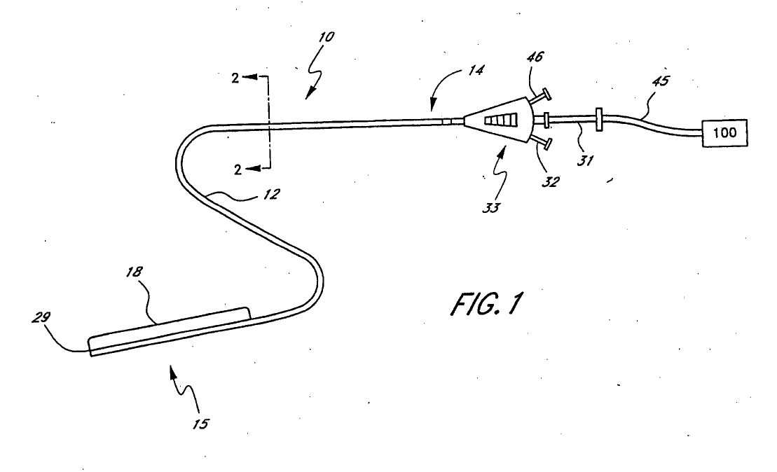

With initial reference to FIG. 1, an ultrasonic catheter 10 configured for use in the large vessels of a patient's anatomy is schematically illustrated. For example, the ultrasonic catheter 10 illustrated in FIG. 1 can be used to treat long segment peripheral arterial occlusions, such as those in the vascular system of the leg.

As illustrated in FIG. 1, the ultrasonic catheter 10 generally comprises a multi-component, elongate flexible tubular body 12 having a proximal region 14 and a distal region 15. The tubular body 12 includes a flexible energy delivery section 18 and a distal exit port 29 located in the distal region 15 of the catheter 10. A backend hub 33 is attached to the proximal region 14 of the tubular body 12, the backend hub 33 comprising a proximal access port 31, an inlet port 32 and a cooling fluid fitting 46. The proximal access port 31 can be connected to control circuitry 100 via cable 45.

The tubular body 12 and other components of the catheter 10 can be manufactured in accordance with any of a variety of techniques well known in the catheter manufacturing field. Suitable materials and dimensions can be readily selected based on the natural and anatomical dimensions of the treatment site and on the desired percutaneous access site.

For example, in a preferred embodiment the proximal region 14 of the tubular body 12 comprises a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 14 of the tubular body 12 is reinforced by braiding, mesh or other constructions to provide increased kink resistance and pushability. For example, nickel titanium or stainless steel wires can be placed along or incorporated into the tubular body 12 to reduce kinking.

In an embodiment configured for treating thrombus in the arteries of the leg, the tubular body 12 has an outside diameter between about 0.060 inches and about 0.075 inches. In another embodiment, the tubular body 12 has an outside diameter of about 0.071 inches. In certain embodiments, the tubular body 12 has an axial length of approximately 105 centimeters, although other lengths may by appropriate for other applications.

The energy delivery section 18 of the tubular body 12 preferably comprises a material that is thinner than the material comprising the proximal region 14 of the tubular body 12 or a material that has a greater acoustic transparency. Thinner materials generally have greater acoustic transparency than thicker materials. Suitable materials for the energy delivery section 18 include, but are not limited to, high or low density polyethylenes, urethanes, nylons, and the like. In certain modified embodiments, the energy delivery section 18 may be formed from the same material or a material of the same thickness as the proximal region 14.

In certain embodiments, the tubular body 12 is divided into at least three sections of varying stiffness. The first section, which preferably includes the proximal region 14, has a relatively higher stiffness. The second section, which is located in an intermediate region between the proximal region 14 and the distal region 15 of the tubular body 12, has a relatively lower stiffness. This configuration further facilitates movement and placement of the catheter 10. The third section, which preferably includes the energy delivery section 18, generally has a lower stiffness than the second section.

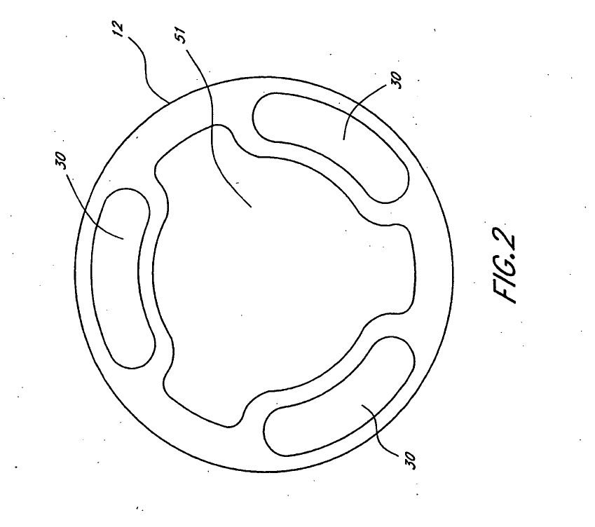

FIG. 2 illustrates a cross section of the tubular body 12 taken along line 2-2 in FIG. 1. In the embodiment illustrated in FIG. 2, three fluid delivery lumens 30 are incorporated into the tubular body 12. In other embodiments, more or fewer fluid delivery lumens can be incorporated into the tubular body 12. The arrangement of the fluid delivery lumens 30 preferably provides a hollow central lumen 51 passing through the tubular body 12. The cross-section of the tubular body 12, as illustrated in FIG. 2, is preferably substantially constant along the length of the catheter 10. Thus, in such embodiments, substantially the same cross-section is present in both the proximal region 14 and the distal region 15 of the catheter 10, including the energy delivery section 18.

In certain embodiments, the central lumen 51 has a minimum diameter greater than about 0.030 inches. In another embodiment, the central lumen 51 has a minimum diameter greater than about 0.037 inches. In one preferred embodiment, the fluid delivery lumens 30 have dimensions of about 0.026 inches wide by about 0.0075 inches high, although other dimensions may be used in other applications.

As described above, the central lumen 51 preferably extends through the length of the tubular body 12. As illustrated in FIG. 1, the central lumen 51 preferably has a distal exit port 29 and a proximal access port 31. The proximal access port 31 forms part of the backend hub 33, which is attached to the proximal region 14 of the catheter 10. The backend hub 33 preferably further comprises cooling fluid fitting 46, which is hydraulically connected to the central lumen 51. The backend hub 33 also preferably comprises a therapeutic compound inlet port 32, which is in hydraulic connection with the fluid delivery lumens 30, and which can be hydraulically coupled to a source of therapeutic compound via a hub such as a Luer fitting.

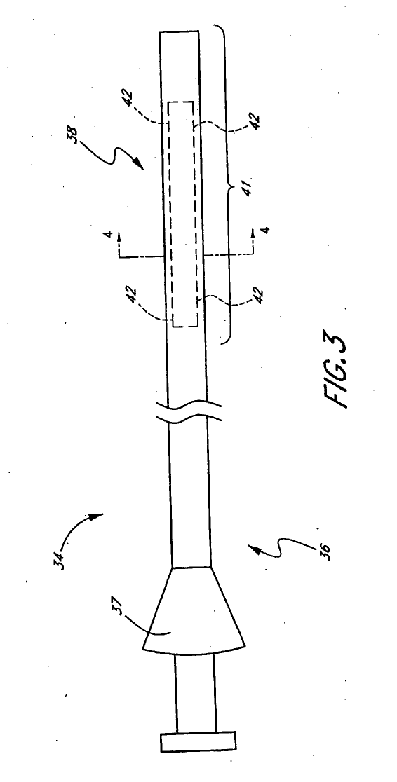

The central lumen 51 is configured to receive an elongate inner core 34 of which a preferred embodiment is illustrated in FIG. 3. The elongate inner core 34 preferably comprises a proximal region 36 and a distal region 38. Proximal hub 37 is fitted on the inner core 34 at one end of the proximal region 36. One or more ultrasound radiating members are positioned within an inner core energy delivery section 41 located within the distal region 38. The ultrasound radiating members form an ultrasound assembly 42, which will be described in greater detail below.

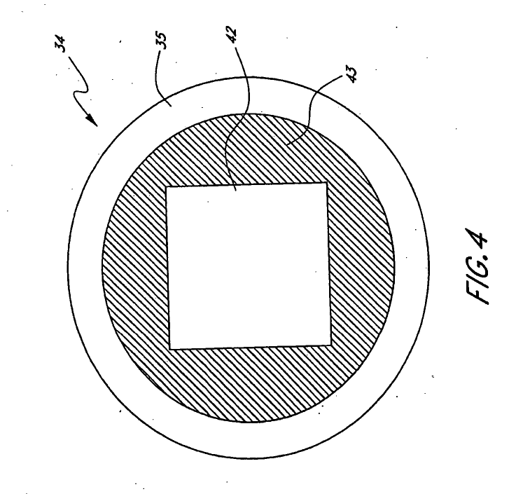

As shown in the cross-section illustrated in FIG. 4, which is taken along lines 4-4 in FIG. 3, the inner core 34 preferably has a cylindrical shape, with an outer diameter that permits the inner core 34 to be inserted into the central lumen 51 of the tubular body 12 via the proximal access port 31. Suitable outer diameters of the inner core 34 include, but are not limited to, about 0.010 inches to about 0.100 inches. In another embodiment, the outer diameter of the inner core 34 is between about 0.020 inches and about 0.080 inches. In yet another embodiment, the inner core 34 has an outer diameter of about 0.035 inches.

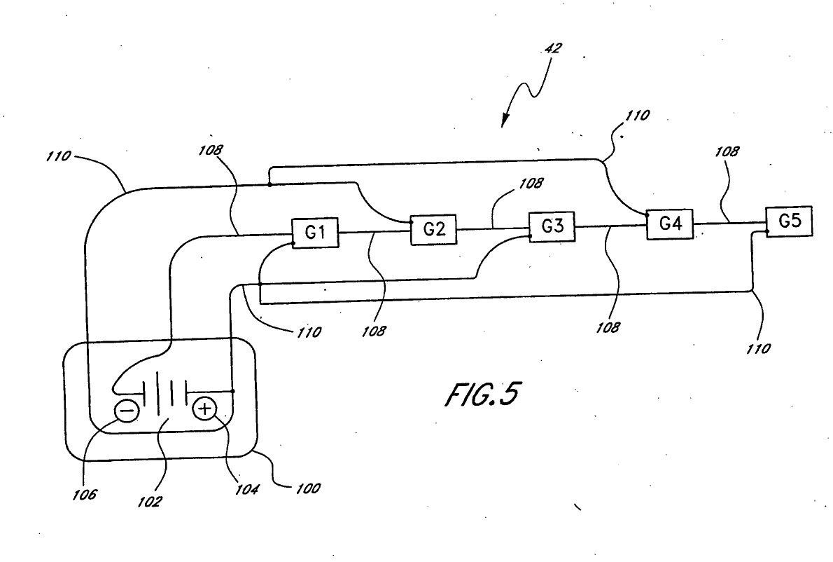

Still referring to FIG. 4, in an exemplary embodiment, the inner core 34 includes a cylindrical outer body 35 that houses the ultrasound assembly 42. The ultrasound assembly 42 comprises wiring and ultrasound radiating members, described in greater detail in FIGS. 5 through 7D, such that the ultrasound assembly 42 is capable of radiating ultrasonic energy from the energy delivery section 41 of the inner core 34. The ultrasound assembly 42 is electrically connected to the backend hub 33, where the inner core 34 can be connected to control circuitry 100 via cable 45 (illustrated in FIG. 1). Preferably, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

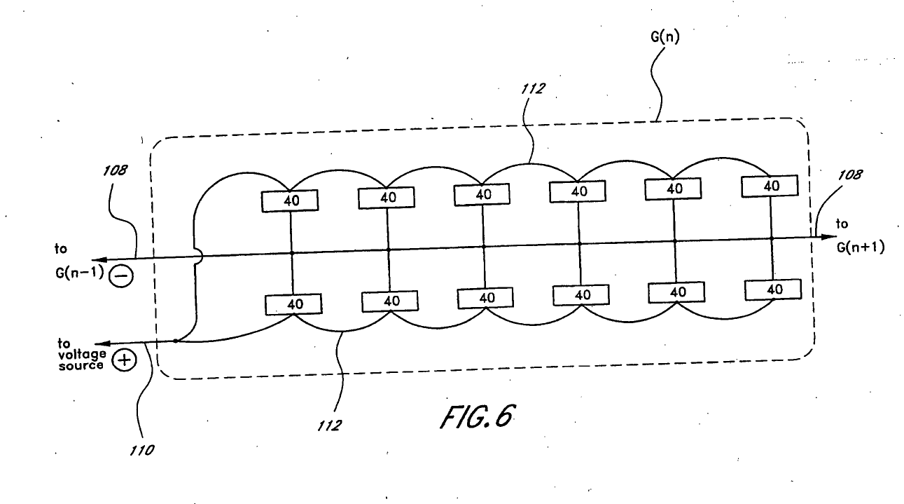

In an exemplary embodiment, the ultrasound assembly 42 comprises a plurality of ultrasound radiating members that are divided into one or more groups. For example, FIGS. 5 and 6 are schematic wiring diagrams illustrating one technique for connecting five groups of ultrasound radiating members 40 to form the ultrasound assembly 42. As illustrated in FIG. 5, the ultrasound assembly 42 comprises five groups G1, G2, G3, G4, G5 of ultrasound radiating members 40 that are electrically connected to each other. The five groups are also electrically connected to the control circuitry 100.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHz. In another embodiment, the waves have a frequency between about 1 MHz and about 3 MHz. In yet another embodiment, the waves have a frequency of about 2 MHz. The average acoustic power is between about 0.01 watts and 300 watts. In one embodiment, the average acoustic power is about 15 watts.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics typically comprise a crystalline material, such as quartz, that change shape when an electrical current is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

Still referring to FIG. 5, the control circuitry 100 preferably comprises, among other things, a voltage source 102. The voltage source 102 comprises a positive terminal 104 and a negative terminal 106. The negative terminal 106 is connected to common wire 108, which connects the five groups G1-G5 of ultrasound radiating members 40 in series. The positive terminal 104 is connected to a plurality of lead wires 110, which each connect to one of the five groups G1-G5 of ultrasound radiating members 40. Thus, under this configuration, each of the five groups G1-G5, one of which is illustrated in FIG. 6, is connected to the positive terminal 104 via one of the lead wires 110, and to the negative terminal 106 via the common wire 108.

Referring now to FIG. 6, each group G1-G5 comprises a plurality of ultrasound radiating members 40. Each of the ultrasound radiating members 40 is electrically connected to the common wire 108 and to the lead wire 110 via one of two positive contact wires 112. Thus, when wired as illustrated, a constant voltage difference will be applied to each ultrasound radiating member 40 in the group. Although the group illustrated in FIG. 6 comprises twelve ultrasound radiating members 40, one of ordinary skill in the art will recognize that more or fewer ultrasound radiating members 40 can be included in the group. Likewise, more or fewer than five groups can be included within the ultrasound assembly 42 illustrated in FIG. 5.

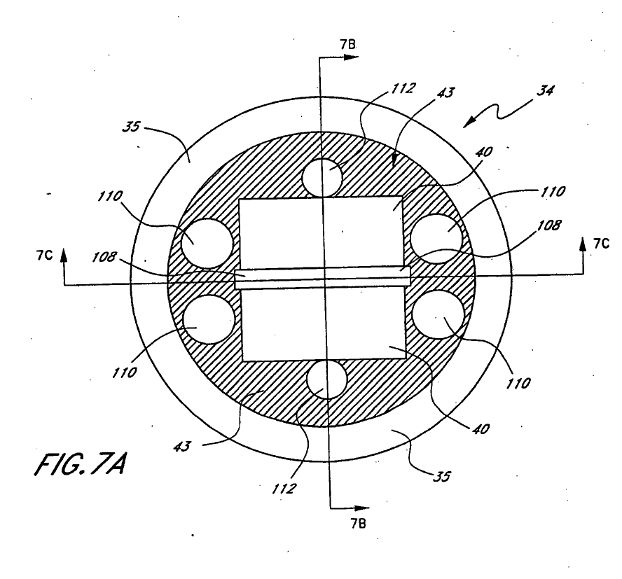

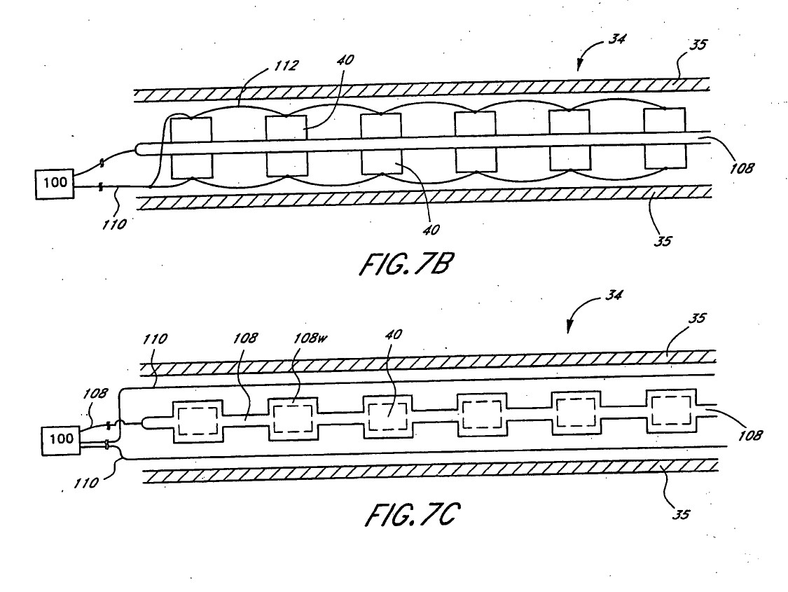

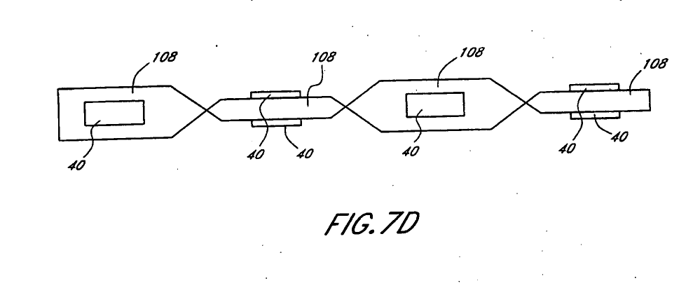

FIG. 7A illustrates one preferred technique for arranging the components of the ultrasound assembly 42 (as schematically illustrated in FIG. 5) into the inner core 34 (as schematically illustrated in FIG. 4). FIG. 7A is a cross-sectional view of the ultrasound assembly 42 taken within group Gl in FIG. 5, as indicated by the presence of four lead wires 110. For example, if a cross-sectional view of the ultrasound assembly 42 was taken within group G4 in FIG. 5, only one lead wire 110 would be present (that is, the one lead wire connecting group G5).

Referring still to FIG. 7A, the common wire 108 comprises an elongate, flat piece of electrically conductive material in electrical contact with a pair of ultrasound radiating members 40. Each of the ultrasound radiating members 40 is also in electrical contact with a positive contact wire 112. Because the common wire 108 is connected to the negative terminal 106, and the positive contact wire 112 is connected to the positive terminal 104, a voltage difference can be created across each ultrasound radiating member 40. Lead wires 110 are preferably separated from the other components of the ultrasound assembly 42, thus preventing interference with the operation of the ultrasound radiating members 40 as described above. For example, in one preferred embodiment, the inner core 34 is filled with an insulating potting material 43, thus deterring unwanted electrical contact between the various components of the ultrasound assembly 42.

FIGS. 7B and 7C illustrate cross sectional views of the inner core 34 of FIG. 7A taken along lines 7B-7B and 7C-7C, respectively. As illustrated in FIG. 7B, the ultrasound radiating members 40 are mounted in pairs along the common wire 108. The ultrasound radiating members 40 are connected by positive contact wires 112, such that substantially the same voltage is applied to each ultrasound radiating member 40. As illustrated in FIG. 7C, the common wire 108 preferably comprises wide regions 108W upon which the ultrasound radiating members 40 can be mounted, thus reducing the likelihood that the paired ultrasound radiating members 40 will short together. In certain embodiments, outside the wide regions 108W, the common wire 108 may have a more conventional, rounded wire shape.

In a modified embodiment, such as illustrated in FIG. 7D, the common wire 108 is twisted to form a helical shape before being fixed within the inner core 34. In such embodiments, the ultrasound radiating members 40 are oriented in a plurality of radial directions, thus enhancing the radial uniformity of the resulting ultrasonic energy field.

The wiring arrangement described above can be modified to allow each group G1, G2, G3, G4, G5 to be independently powered. Specifically, by providing a separate power source within the control system 100 for each group, each group can be individually turned on or off, or can be driven with an individualized power. This provides the advantage of allowing the delivery of ultrasonic energy to be “turned off” in regions of the treatment site where treatment is complete, thus preventing deleterious or unnecessary ultrasonic energy to be applied to the patient.

The embodiments described above, and illustrated in FIGS. 5 through 7, illustrate a plurality of ultrasound radiating members grouped spatially. That is, in such embodiments, all of the ultrasound radiating members within a certain group are positioned adjacent to each other, such that when a single group is activated, ultrasonic energy is delivered at a specific length of the ultrasound assembly. However, in modified embodiments, the ultrasound radiating members of a certain group may be spaced apart from each other, such that the ultrasound radiating members within a certain group are not positioned adjacent to each other. In such embodiments, when a single group is activated, ultrasonic energy can be delivered from a larger, spaced apart portion of the energy delivery section. Such modified embodiments may be advantageous in applications wherein it is desired to deliver a less focussed, more diffuse ultrasonic energy field to the treatment site.

In an exemplary embodiment, the ultrasound radiating members 40 comprise rectangular lead zirconate titanate (“PZT”) ultrasound transducers that have dimensions of about 0.017 inches by about 0.010 inches by about 0.080 inches. In other embodiments, other configurations may be used. For example, disc-shaped ultrasound radiating members 40 can be used in other embodiments. In a preferred embodiment, the common wire 108 comprises copper, and is about 0.005 inches thick, although other electrically conductive materials and other dimensions can be used in other embodiments. Lead wires 110 are preferably 36-gauge electrical conductors, while positive contact wires 112 are preferably 42-gauge electrical conductors. However, one of ordinary skill in the art will recognize that other wire gauges can be used in other embodiments.

As described above, suitable frequencies for the ultrasound radiating member 40 include, but are not limited to, from about 20 kHz to about 20 MHz. In one embodiment, the frequency is between about 500 kHz and 20 MHz, and in another embodiment the frequency is between about 1 MHz and 3MHz. In yet another embodiment, the ultrasound radiating members 40 are operated with a frequency of about 2 MHz.

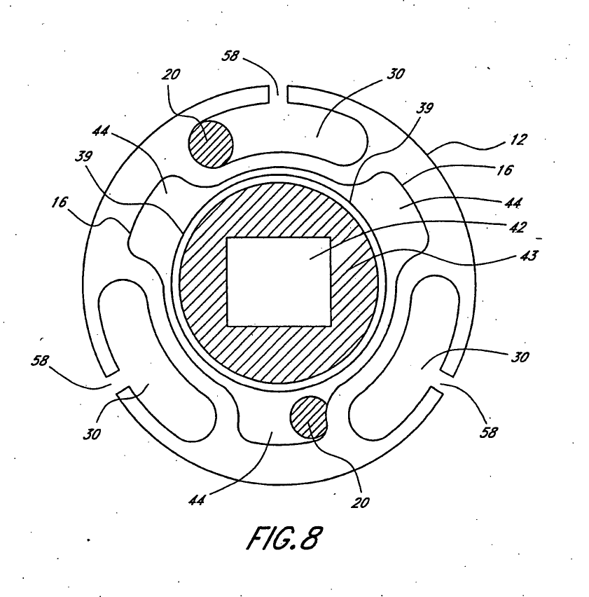

FIG. 8 illustrates the inner core 34 positioned within the tubular body 12. Details of the ultrasound assembly 42, provided in FIG. 7A, are omitted for clarity. As described above, the inner core 34 can be slid within the central lumen 51 of the tubular body 12, thereby allowing the inner core energy delivery section 41 to be positioned within the tubular body energy delivery section 18. For example, in a preferred embodiment, the materials comprising the inner core energy delivery section 41, the tubular body energy delivery section 18, and the potting material 43 all comprise materials having a similar acoustic impedance, thereby minimizing ultrasonic energy losses across material interfaces.

FIG. 8 further illustrates placement of fluid delivery ports 58 within the tubular body energy delivery section 18. As illustrated, holes or slits are formed from the fluid delivery lumen 30 through the tubular body 12, thereby permitting fluid flow from the fluid delivery lumen 30 to the treatment site. Thus, a source of therapeutic compound coupled to the inlet port 32 provides a hydraulic pressure which drives the therapeutic compound through the fluid delivery lumens 30 and out the fluid delivery ports 58.

By evenly spacing the fluid delivery lumens 30 around the circumference of the tubular body 12, as illustrated in FIG. 8, a substantially even flow of therapeutic compound around the circumference of the tubular body 12 can be achieved. In addition, the size, location and geometry of the fluid delivery ports 58 can be selected to provide uniform fluid flow from the fluid delivery lumen 30 to the treatment site. For example, in one embodiment, fluid delivery ports 58 closer to the proximal region of the energy delivery section 18 have smaller diameters than fluid delivery ports 58 closer to the distal region of the energy delivery section 18, thereby allowing uniform delivery of fluid across the entire energy delivery section 18.

For example, in one embodiment in which the fluid delivery ports 58 have similar sizes along the length of the tubular body 12, the fluid delivery ports 58 have a diameter between about 0.0005 inches to about 0.0050 inches. In another embodiment in which the size of the fluid delivery ports 58 changes along the length of the tubular body 12, the fluid delivery ports 58 have a diameter between about 0.001 inches to about 0.005 inches in the proximal region of the energy delivery section 18, and between about 0.005 inches to 0.0020 inches in the distal region of the energy delivery section 18. The increase in size between adjacent fluid delivery ports 58 depends on the material comprising the tubular body 12, and on the size of the fluid delivery lumen 30. The fluid delivery ports 58 can be created in the tubular body 12 by punching, drilling, burning or ablating (such as with a laser), or by any other suitable method. Therapeutic compound flow along the length of the tubular body 12 can also be increased by increasing the density of the fluid delivery ports 58 toward the distal region 15 of the tubular body 12.

It should be appreciated that it may be desirable to provide non-uniform fluid flow from the fluid delivery ports 58 to the treatment site. In such embodiment, the size, location and geometry of the fluid delivery ports 58 can be selected to provide such non-uniform fluid flow.

Referring still to FIG. 8, placement of the inner core 34 within the tubular body 12 further defines cooling fluid lumens 44. Cooling fluid lumens 44 are formed between an outer surface 39 of the inner core 34 and an inner surface 16 of the tubular body 12. In certain embodiments, a cooling fluid is introduced through the proximal access port 31 such that cooling fluid flow is produced through cooling fluid lumens 44 and out distal exit port 29 (see FIG. 1). The cooling fluid lumens 44 are preferably evenly spaced around the circumference of the tubular body 12 (that is, at approximately 120° increments for a three-lumen configuration), thereby providing uniform cooling fluid flow over the inner core 34. Such a configuration is desired to remove unwanted thermal energy at the treatment site. As will be explained below, the flow rate of the cooling fluid and the power to the ultrasound assembly 42 can be adjusted to maintain the temperature of the inner core energy delivery section 41 within a desired range.

In an exemplary embodiment, the inner core 34 can be rotated or moved within the tubular body 12. Specifically, movement of the inner core 34 can be accomplished by maneuvering the proximal hub 37 while holding the backend hub 33 stationary. The inner core outer body 35 is at least partially constructed from a material that provides enough structural support to permit movement of the inner core 34 within the tubular body 12 without kinking of the tubular body 12. Additionally, the inner core outer body 35 preferably comprises a material having the ability to transmit torque. Suitable materials for the inner core outer body 35 include, but are not limited to, polyimides, polyesters, polyurethanes, thermoplastic elastomers and braided polyimides.

In an exemplary embodiment, the fluid delivery lumens 30 and the cooling fluid lumens 44 are open at the distal end of the tubular body 12, thereby allowing the therapeutic compound and the cooling fluid to pass into the patient's vasculature at the distal exit port. Or, if desired, the fluid delivery lumens 30 can be selectively occluded at the distal end of the tubular body 12, thereby providing additional hydraulic pressure to drive the therapeutic compound out of the fluid delivery ports 58. In either configuration, the inner core 34 can prevented from passing through the distal exit port by configuring the inner core 34 to have a length that is less than the length of the tubular body 12. In other embodiments, a protrusion is formed on the inner surface 16 of the tubular body 12 in the distal region 15, thereby preventing the inner core 34 from passing through the distal exit port 29.

In still other embodiments, the catheter 10 further comprises an occlusion device (not shown) positioned at the distal exit port 29. The occlusion device preferably has a reduced inner diameter that can accommodate a guidewire, but that is less than the outer diameter of the central lumen 51. Thus, the inner core 34 is prevented from extending through the occlusion device and out the distal exit port 29. For example, suitable inner diameters for the occlusion device include, but are not limited to, about 0.005 inches to about 0.050 inches. In other embodiments, the occlusion device has a closed end, thus preventing cooling fluid from leaving the catheter 10, and instead recirculating to the proximal region 14 of the tubular body 12. These and other cooling fluid flow configurations permit the power provided to the ultrasound assembly 42 to be increased in proportion to the cooling fluid flow rate. Additionally, certain cooling fluid flow configurations can reduce exposure of the patient's body to cooling fluids.

In certain embodiments, as illustrated in FIG. 8, the tubular body 12 further comprises one or more temperature sensors 20, which are preferably located within the energy delivery section 18. In such embodiments, the proximal region 14 of the tubular body 12 includes a temperature sensor lead wire (not shown) which can be incorporated into cable 45 (illustrated in FIG. 1). Suitable temperature sensors include, but are not limited to, temperature sensing diodes, thermistors, thermocouples, resistance temperature detectors (“RTDs”) and fiber optic temperature sensors which use thermochromic liquid crystals. Suitable temperature sensor 20 geometries include, but are not limited to, a point, a patch or a stripe. The temperature sensors 20 can be positioned within one or more of the fluid delivery lumens 30, and/or within one or more of the cooling fluid lumens 44.

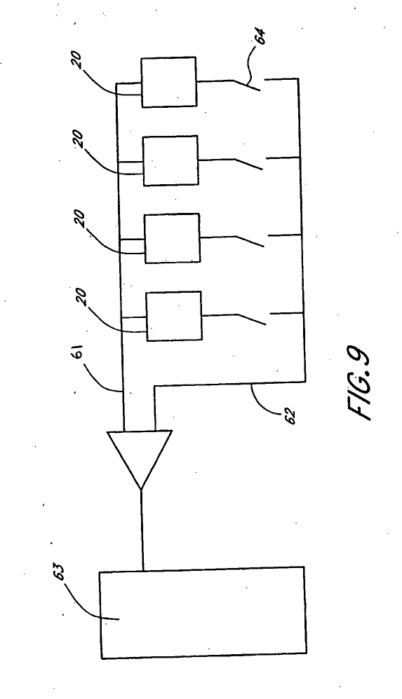

FIG. 9 illustrates one embodiment for electrically connecting the temperature sensors 20. In such embodiments, each temperature sensor 20 is coupled to a common wire 61 and is associated with an individual return wire 62. Accordingly, n+1 wires can be used to independently sense the temperature at n distinct temperature sensors 20. The temperature at a particular temperature sensor 20 can be determined by closing a switch 64 to complete a circuit between that thermocouple's individual return wire 62 and the common wire 61. In embodiments wherein the temperature sensors 20 comprise thermocouples, the temperature can be calculated from the voltage in the circuit using, for example, a sensing circuit 63, which can be located within the external control circuitry 100.

In other embodiments, each temperature sensor 20 is independently wired. In such embodiments, 2n wires pass through the tubular body 12 to independently sense the temperature at n independent temperature sensors 20. In still other embodiments, the flexibility of the tubular body 12 can be improved by using fiber optic based temperature sensors 20. In such embodiments, flexibility can be improved because only n fiber optic members are used to sense the temperature at n independent temperature sensors 20.

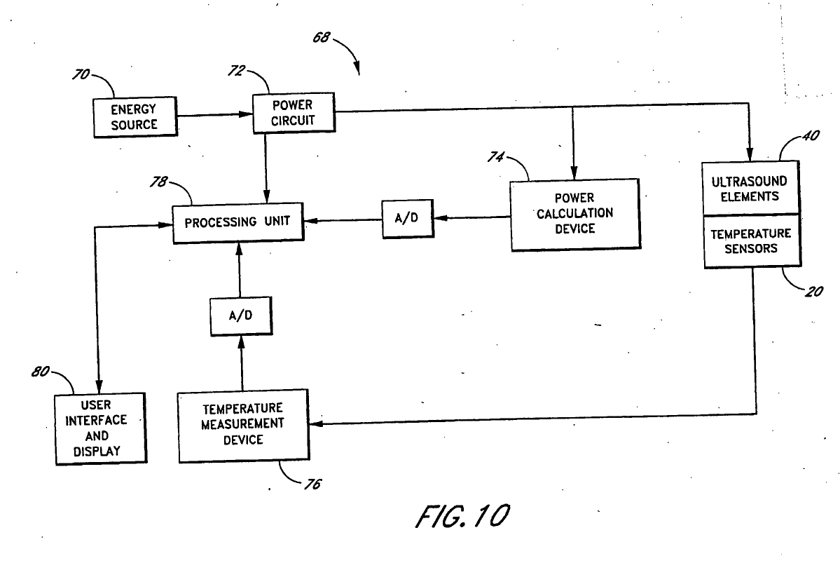

FIG. 10 illustrates one embodiment of a feedback control system 68 that can be used with the catheter 10. The feedback control system 68 can be integrated into the control system that is connected to the inner core 34 via cable 45 (as illustrated in FIG. 1). The feedback control system 68 allows the temperature at each temperature sensor 20 to be monitored and allows the output power of the energy source 70 to be adjusted accordingly. A physician can, if desired, override the closed or open loop system.

The feedback control system 68 preferably comprises an energy source 70, power circuits 72 and a power calculation device 74 that is coupled to the ultrasound radiating members 40. A temperature measurement device 76 is coupled to the temperature sensors 20 in the tubular body 12. A processing unit 78 is coupled to the power calculation device 74, the power circuits 72 and a user interface and display 80.

In operation, the temperature at each temperature sensor 20 is determined by the temperature measurement device 76. The processing unit 78 receives each determined temperature from the temperature measurement device 76. The determined temperature can then be displayed to the user at the user interface and display 80.

The processing unit 78 comprises logic for generating a temperature control signal. The temperature control signal is proportional to the difference between the measured temperature and a desired temperature. The desired temperature can be determined by the user (set at the user interface and display 80) or can be preset within the processing unit 78.

The temperature control signal is received by the power circuits 72. The power circuits 72 are preferably configured to adjust the power level, voltage, phase and/or current of the electrical energy supplied to the ultrasound radiating members 40 from the energy source 70. For example, when the temperature control signal is above a particular level, the power supplied to a particular group of ultrasound radiating members 40 is preferably reduced in response to that temperature control signal. Similarly, when the temperature control signal is below a particular level, the power supplied to a particular group of ultrasound radiating members 40 is preferably increased in response to that temperature control signal. After each power adjustment, the processing unit 78 preferably monitors the temperature sensors 20 and produces another temperature control signal which is received by the power circuits 72.

The processing unit 78 preferably further comprises safety control logic. The safety control logic detects when the temperature at a temperature sensor 20 has exceeded a safety threshold. The processing unit 78 can then provide a temperature control signal which causes the power circuits 72 to stop the delivery of energy from the energy source 70 to that particular group of ultrasound radiating members 40.

Because, in certain embodiments, the ultrasound radiating members 40 are mobile relative to the temperature sensors 20, it can be unclear which group of ultrasound radiating members 40 should have a power, voltage, phase and/or current level adjustment. Consequently, each group of ultrasound radiating member 40 can be identically adjusted in certain embodiments. In a modified embodiment, the power, voltage, phase, and/or current supplied to each group of ultrasound radiating members 40 is adjusted in response to the temperature sensor 20 which indicates the highest temperature. Making voltage, phase and/or current adjustments in response to the temperature sensed by the temperature sensor 20 indicating the highest temperature can reduce overheating of the treatment site.

The processing unit 78 also receives a power signal from a power calculation device 74. The power signal can be used to determine the power being received by each group of ultrasound radiating members 40. The determined power can then be displayed to the user on the user interface and display 80.

As described above, the feedback control system 68 can be configured to maintain tissue adjacent to the energy delivery section 18 below a desired temperature. For example, it is generally desirable to prevent tissue at a treatment site from increasing more than 6° C. As described above, the ultrasound radiating members 40 can be electrically connected such that each group of ultrasound radiating members 40 generates an independent output. In certain embodiments, the output from the power circuit maintains a selected energy for each group of ultrasound radiating members 40 for a selected length of time.

The processing unit 78 can comprise a digital or analog controller, such as for example a computer with software. When the processing unit 78 is a computer it can include a central processing unit (“CPU”) coupled through a system bus. As is well known in the art, the user interface and display 80 can comprise a mouse, a keyboard, a disk drive, a display monitor, a nonvolatile memory system, or any another. Also preferably coupled to the bus is a program memory and a data memory.

In lieu of the series of power adjustments described above, a profile of the power to be delivered to each group of ultrasound radiating members 40 can be incorporated into the processing unit 78, such that a preset amount of ultrasonic energy to be delivered is pre-profiled. In such embodiments, the power delivered to each group of ultrasound radiating members 40 can then be adjusted according to the preset profiles.

The ultrasound radiating members 40 can be operated in a pulsed mode. For example, in one embodiment, the time average power supplied to the ultrasound radiating members 40 is preferably between about 0.1 watts and 2 watts and more preferably between about 0.5 watts and 1.5 watts. In certain preferred embodiments, the time average power is approximately 0.6 watts or 1.2 watts. The duty cycle is preferably between about 1% and 50% and more preferably between about 5% and 25%. In certain preferred embodiments, the duty ratio is approximately 7.5% or 15%. The pulse averaged power is preferably between about 0.1 watts and 20 watts and more preferably between approximately 5 watts and 20 watts. In-certain preferred embodiments, the pulse averaged power is approximately 8 watts and 16 watts. The amplitude during each pulse can be constant or varied.

In one embodiment, the pulse repetition rate is preferably between about 5 Hz and 150 Hz and more preferably between about 10 Hz and 50 Hz. In certain preferred embodiments, the pulse repetition rate is approximately 30 Hz. The pulse duration is preferably between about 1 millisecond and 50 milliseconds and more preferably between about 1 millisecond and 25 milliseconds. In certain preferred embodiments, the pulse duration is approximately 2.5 milliseconds or 5 milliseconds.

In one particular embodiment, the ultrasound radiating members 40 are operated at an average power of approximately 0.6 watts, a duty cycle of approximately 7.5%, a pulse repetition rate of 30 Hz, a pulse average electrical power of approximately 8 watts and a pulse duration of approximately 2.5 milliseconds.

The ultrasound radiating members 40 used with the electrical parameters described herein preferably has an acoustic efficiency greater than 50% and more preferably greater than 75%. The ultrasound radiating members 40 can be formed a variety of shapes, such as, cylindrical (solid or hollow), flat, bar, triangular, and the like. The length of the ultrasound radiating members 40 is preferably between about 0.1 cm and about 0.5 cm. The thickness or diameter of the ultrasound radiating members 40 is preferably between about 0.02 cm and about 0.2 cm.

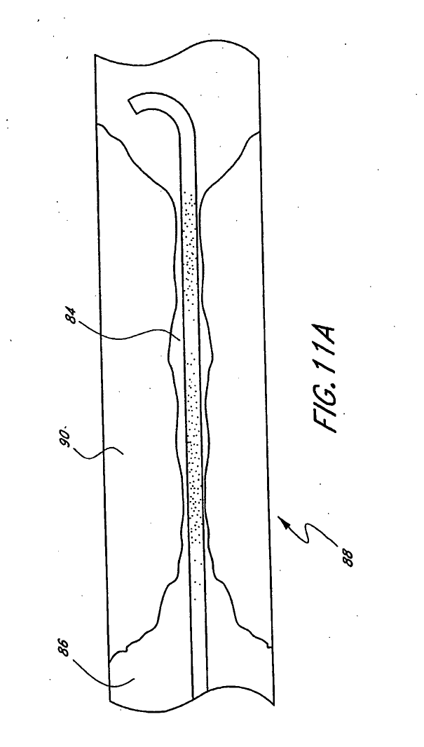

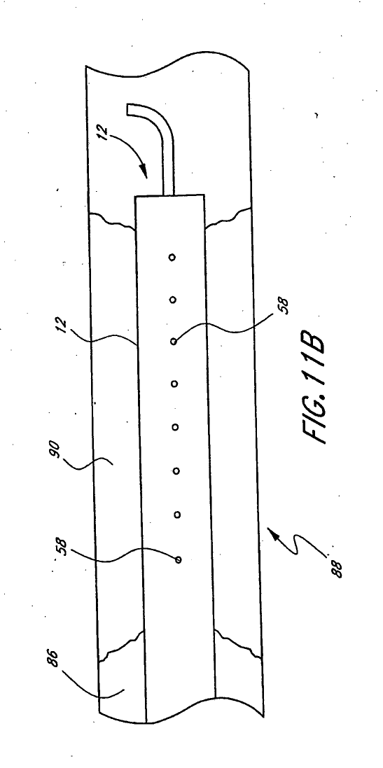

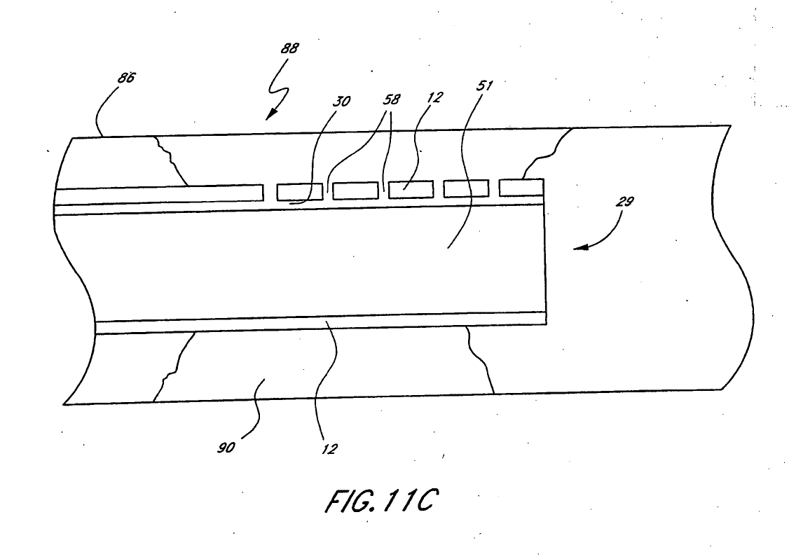

FIGS. 11A through 11D illustrate an exemplary method for using the ultrasonic catheter 10. As illustrated in FIG. 11A, a guidewire 84 similar to a guidewire used in typical angioplasty procedures is directed through a patient's vessels 86 to a treatment site 88 which includes a clot 90. The guidewire 84 is directed through the clot 90. Suitable vessels 86 include, but are not limited to, the large periphery and the small cerebral blood vessels of the body. Additionally, as mentioned above, the ultrasonic catheter 10 also has utility in various imaging applications or in applications for treating and/or diagnosing other diseases in other body parts.

As illustrated in FIG. 11B, the tubular body 12 is slid over and is advanced along the guidewire 84 using conventional over-the-guidewire techniques. The tubular body 12 is advanced until the energy delivery section 18 of the tubular body 12 is positioned at the clot 90. In certain embodiments, radiopaque markers (not shown) are positioned along the energy delivery section 18 of the tubular body 12 to aid in the positioning of the tubular body 12 within the treatment site 88.

As illustrated in FIG. 11C, the guidewire 84 is then withdrawn from the tubular body 12 by pulling the guidewire 84 from the proximal region 14 of the catheter 10 while holding the tubular body 12 stationary. This leaves the tubular body 12 positioned at the treatment site 88.

As illustrated in FIG. 11D, the inner core 34 is then inserted into the tubular body 12 until the ultrasound assembly is positioned at least partially within the energy delivery section 18 of the tubular body 12. Once the inner core 34 is properly positioned, the ultrasound assembly 42 is activated to deliver ultrasonic energy through the energy delivery section 18 to the clot 90. As described above, in one embodiment, suitable ultrasonic energy is delivered with a frequency between about 20 kHz and about 20 MHz.

In a certain embodiment, the ultrasound assembly 42 comprises sixty ultrasound radiating members 40 spaced over a length between approximately 30 cm and 50 cm. In such embodiments, the catheter 10 can be used to treat an elongate clot 90 without requiring movement of or repositioning of the catheter 10 during the treatment. However, it will be appreciated that in modified embodiments the inner core 34 can be moved or rotated within the tubular body 12 during the treatment. Such movement can be accomplished by maneuvering the proximal hub 37 of the inner core 34 while holding the backend hub 33 stationary.

Referring again to FIG. 11D, arrows 48 indicate that a cooling fluid flows through the cooling fluid lumen 44 and out the distal exit port 29. Likewise, arrows 49 indicate that a therapeutic compound flows through the fluid delivery lumen 30 and out the fluid delivery ports 58 to the treatment site 88.

The cooling fluid can be delivered before, after, during or intermittently with the delivery of ultrasonic energy. Similarly, the therapeutic compound can be delivered before, after, during or intermittently with the delivery of ultrasonic energy. Consequently, the steps illustrated in FIGS. 11A through 11D can be performed in a variety of different orders than as described above. In an exemplary embodiment, the therapeutic compound and ultrasonic energy are applied until the clot 90 is partially or entirely dissolved. Once the clot 90 has been dissolved to the desired degree, the tubular body 12 and the inner core 34 are withdrawn from the treatment site 88.

Overview of a Small Vessel Ultrasound Catheter.

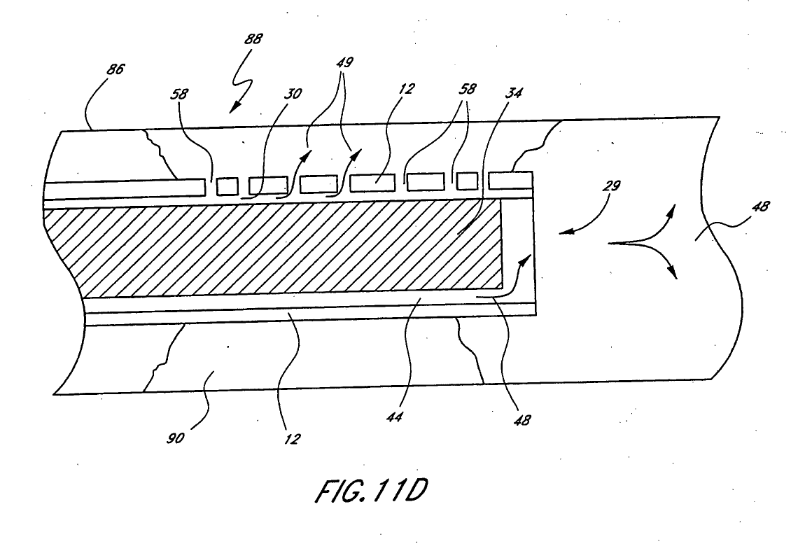

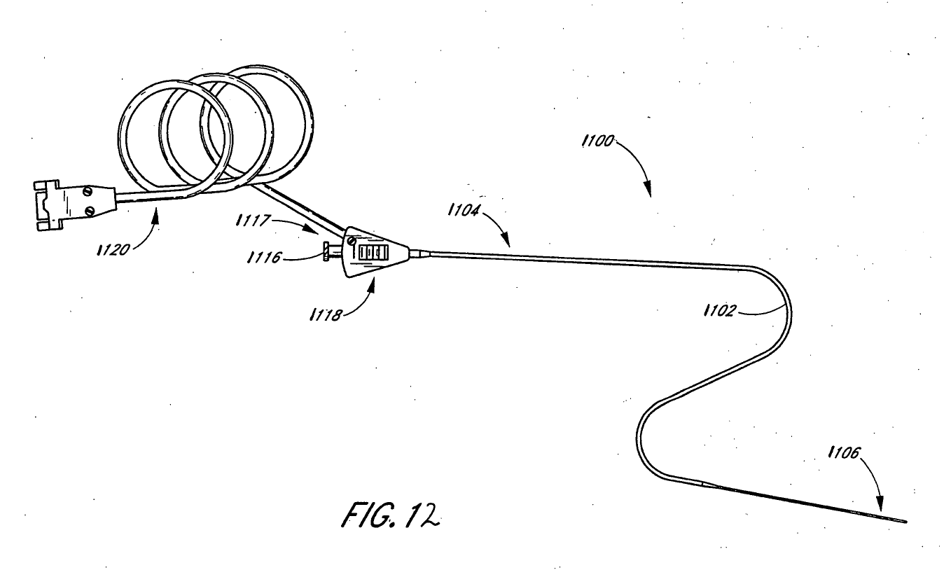

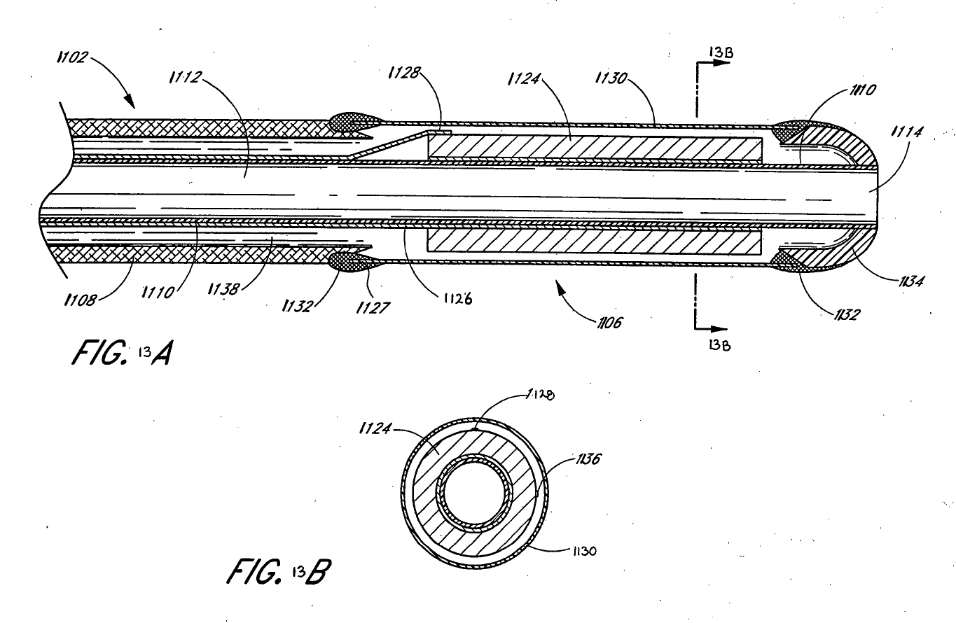

FIGS. 12 through 13B illustrate an exemplary embodiment of an ultrasound catheter 1100 that is well suited for use within small vessels of the distal anatomy, such as the remote, small diameter blood vessels located in the brain.

As shown in FIG. 12 and 13A, the ultrasound catheter 1100 generally comprises a multi-component tubular body 1102 having a proximal end 1104 and a distal end 1106. The tubular body 1102 and other components of the catheter 1100 can be manufactured in accordance with any of a variety of techniques well known in the catheter manufacturing field. As discussed in more detail below, suitable materials and dimensions can be readily selected taking into account the natural and anatomical dimensions of the treatment site and of the desired percutaneous access site.

The tubular body 1102 can be divided into multiple sections of varying stiffness. For example, a first section, which includes the proximal end 1104, is generally more stiff than a second section, which lies between the proximal end 1104 and the distal end 1106 of the tubular body 1102. This arrangement facilitates the movement and placement of the ultrasound catheter 1100 within small vessels. A third section, which includes at least one ultrasound radiating member 1124, is generally stiffer than the second section due to the presence of the ultrasound radiating member 1124.

In the exemplary embodiments described herein, the assembled ultrasound catheter has sufficient structural integrity, or “pushability,” to permit the catheter to be advanced through a patient's vasculature to a treatment site without significant buckling or kinking. In addition, in certain embodiments, the catheter can transmit torque (that is, the catheter has “torqueability”), thereby allowing the distal portion of the catheter to be rotated into a desired orientation by applying a torque to the proximal end.

Referring now to FIG. 13A, the elongate flexible tubular body 1102 comprises an outer sheath 1108 positioned upon an inner core 1110. In an embodiment particularly well suited for small vessels, the outer sheath 1108 comprises a material such as extruded PEBAXS, polytetrafluoroethylene (“PTFE”), polyetheretherketone (“PEEK”), polyethylene (“PE”), polyimides, braided and/or coiled polyimides and/or other similar materials. The distal end portion of the outer sheath 1108 is adapted for advancement through vessels having a small diameter, such as found in the brain. In an exemplary embodiment, the distal end portion of the outer sheath 1108 has an outer diameter between about 2 French and about 5 French. In another exemplary embodiment, the distal end portion of the outer sheath 1108 has an outer diameter of about 2.8 French. In an exemplary embodiment, the outer sheath 1108 has an axial length of approximately 1150 centimeters. In other embodiments, other dimensions can be used.

In other embodiments, the outer sheath 1108 can be formed from a braided and/or coiled tubing comprising, for example, high or low density polyethylenes, urethanes, nylons, and so forth. Such a configuration enhances the flexibility of the tubular body 1102. For enhanced pushability and torqueability, the outer sheath 1108 can be formed with a variable stiffness from the proximal to the distal end. To achieve this, a stiffening member can be included along the proximal end of the tubular body 1102. In one exemplary embodiment, the pushability and flexibility of the tubular body 1102 are controlled by manipulating the material and thickness of the tubular body 1102, while the torqueability, kink resistance, distortion (also referred to as “ovalization”) and burst strength of the tubular body 1102 are controlled by incorporation of braiding and/or coiling along or into the tubular body 1102.

The inner core 1110 at least partially defines a delivery lumen 1112. In an exemplary embodiment, the delivery lumen 1112 extends longitudinally along substantially the entire length of the ultrasound catheter 1100. The delivery lumen 1112 comprises a distal exit port 1114 and a proximal access port 1116. Referring again to FIG. 12, the proximal access port 1116 is defined by therapeutic compound inlet port 1117 of backend hub 1118, which is attached to the proximal end 104 of the tubular body 1102. In an exemplary embodiment, the illustrated backend hub 1118 is attached to a control box connector 1120. In a modified embodiment, electronics and/or control circuitry for controlling the ultrasound radiating member are incorporated into the backend hub 1118.

In an exemplary embodiment, the delivery lumen 1112 is configured to receive a guide wire (not shown). In one embodiment, the guidewire has a diameter of approximately 0.008 inches to approximately 0.020 inches. In another embodiment, the guidewire has a diameter of about 0.014 inches. In an exemplary embodiment, the inner core 1110 comprises polyimide or a similar material which, in some embodiments, can be braided and/or coiled to increase the flexibility of the tubular body 1102.

Referring now to the exemplary embodiment illustrated in FIGS. 13A and 13B, the distal end 1106 of the tubular body 1102 includes an ultrasound radiating member 1124. In an exemplary embodiment, the ultrasound radiating member 1124 comprises an ultrasound transducer that converts, for example, electrical energy into ultrasonic energy. In a modified embodiment, the ultrasonic energy can be generated by an ultrasound transducer that is remote from the ultrasound radiating member 1124, and the ultrasonic energy can be transmitted via, for example, a wire to the ultrasound radiating member 1124.

As illustrated in FIGS. 13A and 13B, the ultrasound radiating member 1124 is configured as a hollow cylinder. As such, the inner core 1110 extends through the hollow core of the ultrasound radiating member 1124. In an exemplary embodiment, the ultrasound radiating member 1124 is secured to the inner core 1110 in a suitable manner, such as with an adhesive. A potting material can also be used to help secure the ultrasound radiating member 1124 to the inner core 1110.

In other embodiments, the ultrasound radiating member 1124 has a different shape. For example, the ultrasound radiating member 1124 can be shaped as a solid rod, a disk, a solid rectangle or a thin block. In still other embodiments, the ultrasound radiating member 1124 comprises a plurality of smaller ultrasound radiating elements. The embodiments illustrated in FIGS. 12 through 13B advantageously provide enhanced cooling of the ultrasound radiating member 1124. For example, in an exemplary embodiment, a therapeutic compound is delivered through the delivery lumen 1112. As the therapeutic compound passes through the central core of the ultrasound radiating member 1124, the therapeutic compound advantageously removes heat generated by the ultrasound radiating member 1124. In another embodiment, a return path can be formed in region 1138 between the outer sheath 1108 and the inner core 1110 such that coolant from a coolant system passes through region 1138.

In an exemplary embodiment, the ultrasound radiating member 1124 is selected to produce ultrasonic energy in a frequency range adapted for a particular application. Suitable frequencies of ultrasonic energy for the applications described herein include, but are not limited to, from about 20 kHz to about 20 MHz. In one embodiment, the frequency is between about 500 kHz and about 20 MHz, and in another embodiment, the frequency is between about 1 MHz and about 3 MHz. In yet another embodiment, the ultrasonic energy has a frequency of about 3 MHz. In one embodiment, the dimensions of the ultrasound radiating member 1124 are selected to allow the germination of sufficient acoustic energy to enhance lysis without significantly adversely affecting catheter maneuverability.

As described above, in the embodiment illustrated in FIGS. 12 through 13B, ultrasonic energy is generated from electrical power supplied to the ultrasound radiating member 1124. The electrical power can be supplied through control box connector 1120, which is connected to conductive wires 1126, 1128 that extend through the tubular body 1102. In another embodiment, the electrical power can be supplied from a power supply contained within the backend hub 1118. In such embodiments, the conductive wires 1126, 1128 can be secured to the inner core 1110, can lay along the inner core 1110, and/or can extend freely in the region 1138 between the inner core 1110 and the outer sheath 1108. In the illustrated embodiments, the first wire 1126 is connected to the hollow center of the ultrasound radiating member 1124, while the second wire 1128 is connected to the outer periphery of the ultrasound radiating member 1124. In an exemplary embodiment, the ultrasound radiating member 1124 comprises a transducer formed of a piezoelectric ceramic oscillator or a similar material.

In the exemplary embodiment illustrated in FIGS. 13A and 13B, the distal end 1106 of the tubular body 1102 includes a sleeve 1130 that is generally positioned about the ultrasound radiating member 1124. In such embodiments, the sleeve, 1130 comprises a material that readily transmits ultrasonic energy. Suitable materials for the sleeve 130 include, but are not limited to, polyolefins, polyimides, polyesters and other materials that readily transmit ultrasonic energy with minimal energy absorption. In an exemplary embodiment, the proximal end of the sleeve 1130 is attached to the outer sheath 1108 with an adhesive 1132. In certain embodiments, to improve the bonding of the adhesive 1132 to the outer sheath 1108, a shoulder 1127 or notch is formed in the outer sheath 1108 for attachment of the adhesive 1132 thereto. In an exemplary embodiment, the outer sheath 1108 and the sleeve 1130 have substantially the same outer diameter. In other embodiments, the sleeve 1130 can be attached to the outer sheath 1108 using heat bonding techniques, such as radiofrequency welding, hot air bonding, or direct contact heat bonding. In still other embodiments, techniques such as over molding, dip coating, film casting and so forth can be used.

Still referring to the exemplary embodiment illustrated in FIGS. 13A and 13B, the distal end of the sleeve 1130 is attached to a tip 1134. As illustrated, the tip 1134 is also attached to the distal end of the inner core 1110. In one embodiment, the tip is between about 0.5 millimeters and about 4.0 millimeters long. In another embodiment, the tip is about 2.0 millimeters long. As illustrated, in certain embodiments the tip is rounded in shape to reduce trauma or damage to tissue along the inner wall of a blood vessel or other body structure during advancement toward a treatment site.

As illustrated in FIG. 13B, the ultrasound catheter 1100 can include at least one temperature sensor 1136 in the distal region of the catheter. In one embodiment, the temperature sensor 1136 is positioned on or near the ultrasound radiating member 1124. Suitable temperature sensors include but are not limited to, diodes, thermistors, thermocouples, resistance temperature detectors, and fiber optic temperature sensors that use thermochromic liquid crystals. In an exemplary embodiment, the temperature sensor 1136 is operatively connected to a control box (not shown) through a control wire that extends along the tubular body 1102 and through the backend hub 1118, and that is operatively connected to the control box via control box connector 1120. In an exemplary embodiment, the control box includes a feedback control system having the ability to monitor and control the power, voltage, current and phase supplied to the ultrasound radiating member 1124. In this manner, the temperature along a selected region of the ultrasound catheter 1100 can be monitored and controlled. Details of the control box can be found in U.S. patent application Publication 2004/0024347 (published 5 Feb. 2004) and U.S. patent application Publication 2004/0049148 (published 11 Mar. 2004), which are both incorporated by reference herein in their entirety.

In embodiments wherein multiple ultrasound radiating members are positioned in the catheter distal region, a plurality of temperature sensors can be positioned adjacent to the ultrasound radiating members. For example, in one such embodiment, a temperature sensor is positioned on or near each of the multiple ultrasound radiating members.

In an exemplary application, the ultrasound catheter 1100 can be used to remove an occlusion from a small blood vessel. In such an exemplary application, a free end of a guidewire is percutaneously inserted into a patient's vasculature at a suitable first puncture site. The guidewire is advanced through the vasculature toward a treatment site where the blood vessel is occluded by a thrombus. In one embodiment, the guidewire is directed through the thrombus. In another embodiment, the guidewire is directed through the thrombus, and is left in the thrombus during treatment to aid in dispersion of the therapeutic compound into the thrombus.

After advancing the guidewire to the treatment site, the ultrasound catheter 1100 is percutaneously inserted into the patient's vasculature through the first puncture site, and is advanced along the guidewire towards the treatment site using conventional over-the-guidewire techniques. The ultrasound catheter 1100 is advanced until the distal end is positioned at or within the occlusion. In a modified embodiment, the catheter distal end includes one or more radiopaque markers (not shown) to aid in positioning the catheter distal end at the treatment site.

After the ultrasound catheter 1100 is positioned, the guidewire can be withdrawn from the delivery lumen 1112. A therapeutic compound source (not shown), such as a syringe with a Luer fitting, is hydraulically connected to the therapeutic compound inlet port 1117, and the control box connector 1120 is connected to the control box. This configuration allows a therapeutic compound to be delivered through the delivery lumen 1112 and the distal exit port 1114 to the occlusion. One exemplary therapeutic compound appropriate for treating a thrombus is an aqueous solution containing heparin, urokinase, streptokinase, and/or tissue plasminogen activator.

The ultrasound radiating member 1124 can be activated to emit ultrasonic energy from the distal region of the ultrasound catheter 1100. As described above, suitable frequencies for the ultrasonic energy include, but are not limited to, from about 20 kHz to about 20 MHz. In one embodiment, the frequency is between about 500 kHz and about 20 MHz, and in another embodiment the frequency is between about 1 MHz and 3 MHz. In yet another embodiment, the ultrasonic energy has a frequency of about 3 MHz. In an exemplary embodiment, the therapeutic compound and ultrasonic energy are applied until the thrombus is partially or entirely dissolved. Once the thrombus has been dissolved sufficiently, the ultrasound catheter 1100 is withdrawn from the treatment site.

The catheters described herein can be manufactured by sequentially positioning the various catheter components onto the catheter assembly. For example, in one method of manufacture, the ultrasound radiating member 1124 is positioned over the outer surface of an intermediate portion of an elongate tube. The elongate tube serves as the inner core 1110 and defines the delivery lumen 1112. The first and second wires 1126, 1128 are then also disposed along the outer surface of the inner core 1110 proximal to the ultrasound radiating member 1124. The first wire 1126 is electrically connected to an inner surface of the ultrasound radiating member 1124, and the second wire is electrically connected to an outer surface of the ultrasound radiating member 1124, as illustrated in FIG. 13A. The electrical connections can be accomplished using, for example, a solder joint.

After the ultrasound radiating member 1124 and wires 1126, 1128 are secured to the inner core 1110, an outer sheath 1108 is positioned over a portion of the inner core, leaving the ultrasound radiating member 1124 uncovered by the outer sheath 1108, as illustrated in FIG. 13A. A cylindrical sleeve 1130 is then positioned over the ultrasound radiating member 1124, and is secured to the distal end of the outer sheath 1108 with an adhesive 1132. A rounded distal tip 1134 is then secured to the sleeve 1130 and the inner core 1110, and any excess length of the elongate tube extending distal to the distal tip 1134 is removed.

Although an exemplary catheter manufacturing technique has been expounded above, other manufacturing techniques can be used, additional components can be included, and the components set forth above can be modified. For example, in certain embodiments, the ultrasound catheter 1100 further comprises a temperature sensor 1136 positioned near the ultrasound radiating member 1124, as described above. In other embodiments, the outer sheath 1108 can be modified to manipulate the flexibility of the catheter 1100, such as by including a stiffening component or metallic braiding and/or coiling.

Overview of a Catheter With Ultrasound-Controllable Porous Membrane.

As described herein, catheters and catheter structures, such as balloons, are made of a thin-walled plastic tubing in certain embodiments. In a modified embodiment, the thin-walled plastic tubing is made semi-porous by forming micro-holes in the catheter tubing. Micro-holes can be formed, for example, by a polymerization process control, or by casting over micro-hole molds.

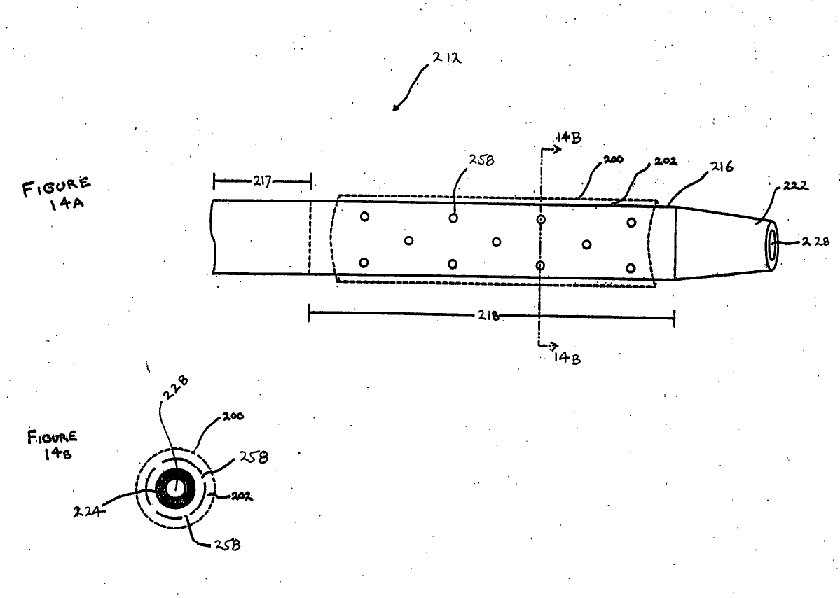

FIGS. 14A and 14B illustrate the distal end of an exemplary ultrasound catheter having an elongate flexible body 212 that includes a support section 217 and an energy delivery section 218. A utility lumen 228 extends through the catheter, and an occlusion device 222 is positioned at the distal end of the catheter. The catheter also includes therapeutic compound delivery ports 258 and a membrane 200 with ultrasound-controllable porosity. In such embodiments, the membrane 200 is cast or formed as a tube, similar to catheter tubing. In other embodiments, the membrane 200 is tightly fit around the catheter, such that the gap 202 between the membrane 200 and the outer sheath 216 does not exist. Transmission of substances of a known mass or size across such membranes is controllable by application of ultrasonic energy from the ultrasonic radiating member 224.

More specifically, exposing the membrane 200 to ultrasonic energy with a predefined frequency and power density will cause certain substances (for example, therapeutic compounds) to pass through the membrane 200. By subsequently switching off the ultrasonic radiating member 224, the porosity of the membrane 200 can be reduced by a factor of approximately 0.5 to approximately 0.001. Thus, this configuration causes delivery of a therapeutic compound to occur mostly and in some embodiments only in the regions of the catheter where ultrasonic energy irradiates the membrane 200.

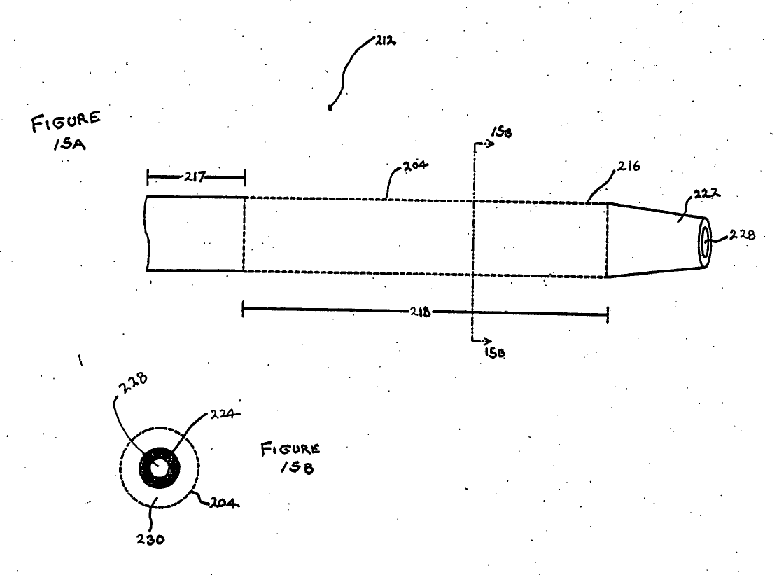

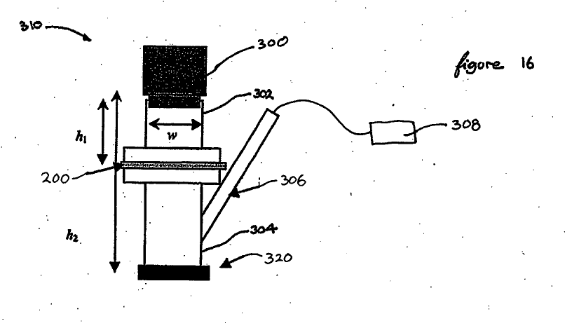

As illustrated in FIGS. 15A and 15B, in other embodiments the outer sheath 216 is at least partially comprised of a material with ultrasound-controllable porosity in region 204. In such embodiments, when ultrasonic energy is emitted from the ultrasound radiating member 224, the outer sheath 216 becomes permeable in the region of the ultrasonic energy emission. This change in permeability permits a therapeutic compound within a therapeutic compound delivery member 230 to pass through the outer sheath 216.

In other embodiments, the tubular body 12 (see FIG. 2) comprises a material with ultrasound-controllable porosity. In such embodiments, when a region of the tubular body 12 is exposed to ultrasonic energy, therapeutic compound will flow out of the fluid delivery lumens 30 in that region. In this configuration, the fluid delivery ports 58 are optional. In still other embodiments, the membrane 200 can be positioned over the distal exit port of an ultrasound catheter, such as the distal exit port 1114 illustrated in FIG. 13A.

Materials with “ultrasound-controllable porosity” refers to a material having a porosity that changes when exposed to ultrasonic energy. Such materials include, but are not limited to, Teflon®, urethanes, silicones, or other materials commonly used in catheter manufacture.

For example, in one embodiment, the membrane 200 with ultrasound-controllable porosity comprises a polycarbonate membrane, available from Millipore (Billerica, Mass.). Sheets of polycarbonate membranes having various pore sizes are readily available and offer a well-controlled medium to assess the effect of ultrasonic energy on solute diffusion. Additionally, polycarbonate membranes have particularly straight and uniform cylindrical holes. In an exemplary embodiment, polycarbonate membranes having the following characteristics are used:

| Characteristic | Approximate Value |
|---|---|
| Pore Size | 10 nm to 10 μm |
| Porosity | 10⁶ to 10⁸ pores cm⁻² |
| Total Pore Area | 0.02% to 0.2% |
| Thickness | 6 to 14 μm |
| Nominal Tare Mass | 1.0 mg cm⁻² |
| Specific Gravity | 0.94 to 0.97 |
| Tensile Strength | <3000 lb in² (207 bar) |
| Autoclavable | yes |
| Leachables | negligible |
| Wetting Characteristics | hydrophilic |
| Maximum Service Temperature | 140° C. (280° F.) |
| Optical Properties | translucent |

In another exemplary embodiment, the membrane 200 with ultrasound-controllable porosity comprises a dialysis membrane, available from Fisher Scientific (Hampton, N.H.). Dialysis membranes are available in various molecular weight cutoffs ranging from 100 Da to 300,000 Da, and thus offer a close match between pore size and solute size. In certain embodiments, ultrasonic energy has a particularly strong effect on transmembrane diffusion when the solute size is approximately equal to the membrane pore size.

Those of skill in the art will recognize that it may be advantageous to test or monitor the properties of the membrane having ultrasound-controllable porosity in a laboratory setting before applying it to catheter. In this manner, through routine experimentation, the optimum membrane properties may be chosen for achieving a desired porosity as a function of ultrasound frequency and/or intensity. As such, an exemplary experimental configuration for determining the porosity of a membrane as a function of ultrasonic frequency, intensity and other factors will now be described.

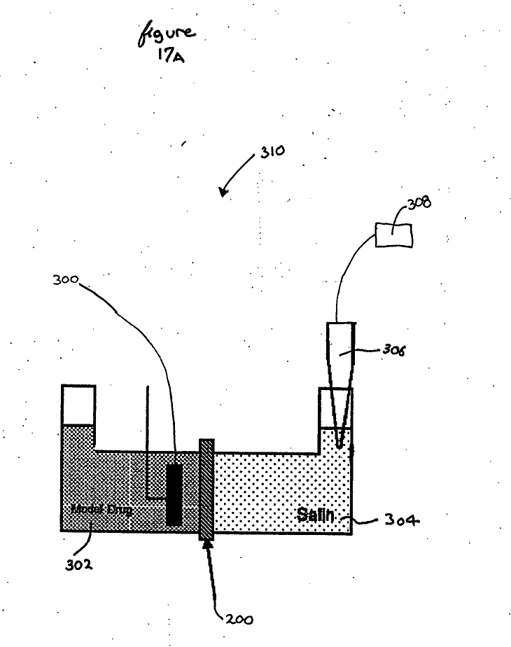

FIG. 16 is an exemplary apparatus 310 for such experimentation. As described below, this configuration is useful for such monitoring. In one embodiment, a hydrophilic solute having a molecular weight between 10³ Da and 10⁶ Da is delivered through a membrane having ultrasound-controllable porosity. An example of such a hydrophilic solute is dextran. In one embodiment, the solute is radiolabeled (³H), thereby allowing solute concentration on at least one side of the membrane to be monitored using a scintillation counter. In other embodiments, other solutes having different physical properties are used.

As shown in FIG. 16, the exemplary apparatus 310 is configured for laboratory monitoring of the properties of a membrane having ultrasound-controllable porosity. The apparatus 310 comprises a external transducer 300 and a horizontally-oriented membrane 200 having ultrasound-controllable porosity. The transducer 300 is separated from the membrane 200 by donor compartment 302. Donor compartment 302 has a height h₁. In one embodiment, height h₁ is between approximately 0.25 cm and approximately 4.0 cm, and in another embodiment, height h₁ is between approximately 0.5 cm and approximately 1.5 cm. In yet another embodiment, the donor compartment 302 has a height h₁ that is approximately 1.0 cm. Donor compartment 302 has a width w. In one embodiment, width w is between approximately 1.0 cm and approximately 9.0 cm, and in another embodiment, width w is between approximately 2.0 cm and approximately 4.0 cm. In yet another embodiment, the donor compartment 302 has a width w that is approximately 3.0 cm. In an exemplary embodiment, the external transducer 300 is Model TL-03, available from EKOS Corporation (Bothell, Wash.).

The laboratory monitoring apparatus 310 illustrated in FIG. 16 further comprises an ultrasound absorber 320. The ultrasound absorber 320 is separated from the membrane 200 by a receiver compartment 304. Receiver compartment 304 and donor compartment 302 have a combined height h₂, which is approximately equal to the distance between the transducer 300 and the ultrasound absorber 320. In one embodiment, height h₂ is between approximately 1.0 cm and 16 cm, and in another embodiment, height h₂ is between approximately 3.0 cm and 5.0 cm. In yet another embodiment, height h₂ is approximately 4.0 cm.

The ultrasound absorber 320 is configured to prevent standing waves from forming within the donor compartment 302 and the receiver compartment 304. In an exemplary embodiment, receiver compartment 304 is outfitted with sampling port 306 connected to a scintillation counter 308 for measuring the concentration of a radiolabeled solute present in the receiver compartment 304.

Referring still to FIG. 16, in an exemplary method for laboratory monitoring of the properties of the membrane 200 with ultrasound-controllable porosity, the donor compartment 302 are the receiver compartment 304 are first filled with a common solution. In one embodiment, the common solution is prepared with air equilibrated tap water (after approximately two days), and is stirred and warmed to approximately 37° C. In other embodiments, the common solution is left at room temperature. The temperature of the membrane 200 is measured Frequently, and, in an exemplary embodiment, does not exceed 43° C. By placing a solute in the donor compartment 302, exposing the membrane 200 to ultrasonic energy, and measuring the presence of the solute in the receiver compartment 304, the ultrasound-controllable porosity of the membrane 200 can be determined.

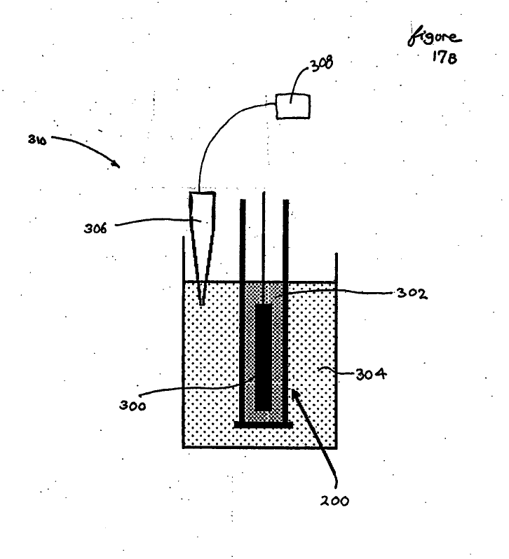

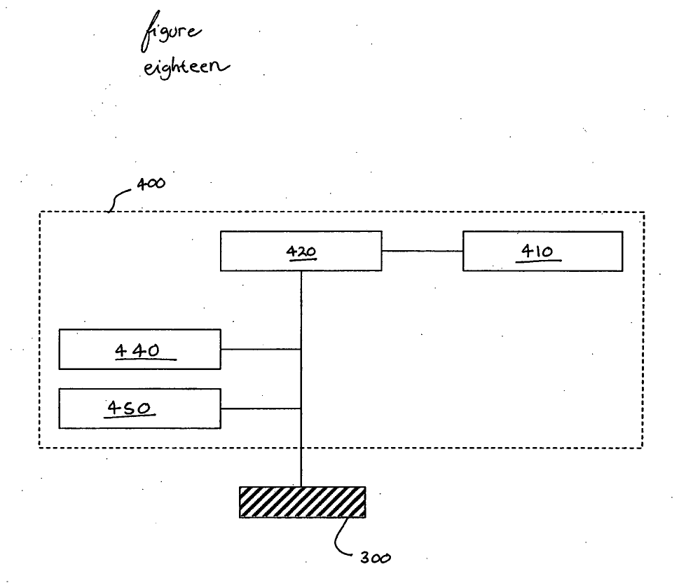

Modified embodiments the laboratory monitoring apparatus 310 are illustrated in FIGS. 17A and 17B. The apparatuses 310 illustrated in FIGS. 17A and 17B include a vertically-oriented membrane 200 that separates a donor compartment 302 from a receiver compartment 304. An ultrasound radiating member 300 is positioned proximal to the vertically-oriented membrane 200. The receiver compartment 304 preferably further comprises a sampling port 306 connected to a scintillation counter 308 for measuring the concentration of radiolabeled solute present in the receiver compartment 304. The experimental methods described herein for use with the apparatus illustrated in FIG. 16 can also be used with the apparatuses illustrated in FIGS. 17A and 17B.

An exemplary configuration for the driving electronics 400 for the laboratory monitoring apparatuses 310 is illustrated in FIG. 18. Such driving electronics 400 can be used with the embodiments illustrated in FIGS. 16 through 17B, or with other similar embodiments. Driving electronics 400 comprise a signal generator 410 which creates a driving signal which is amplified by amplifier 420. The amplified driving signal is then passed to ultrasound radiating member 300. Wattmeter 440 and oscilloscope 450 monitor the power and other characteristics of the amplified driving signal passed to the ultrasound radiating member 300.

In certain embodiments, an experimental setup comprises evaluating two different membranes and four different solutes:

| | |
|---|---|
| Membrane A | polycarbonate with 10 nm pore size |
| Membrane B | cellulose with 100 kDa pore size |
| Solute A | dextran |
| | molecular weight 10³ Da |
| | approximate molecular diameter 1.2 nm |
| Solute B | dextran |
| | molecular weight 10⁴ Da |
| | approximate molecular diameter 2.5 nm |
| Solute C | dextran |
| | molecular weight 10⁵ Da |
| | approximate molecular diameter 6.0 nm |
| Solute D | dextran |
| | molecular weight 10⁶ Da |
| | approximate molecular diameter 11 nm |

This experimental setup permits determination of molecular weight cutoffs for the membranes under study. Additionally, because permeation is inversely proportional to the concentration gradient across the membrane, the maximum reasonable solute concentration can be determined.

The following experimental protocol has been proven especially efficient for ultrasound-enhanced thrombolysis, and thus is used in an exemplary embodiment:

| Characteristic | Value | Range |
|---|---|---|
| Frequency | 2.1 MHz | |
| Duty Cycle | 7.5% | 1% to 100% |
| Average Power | 0.45 W | |
| Pulse Repetition Frequency | 30 Hz | 1 Hz to 10 kHz |
| Time Average Acoustic Energy | ˜5 W cm⁻² | 0.5 to 40 W cm⁻² |
| Peak Acoustic Pressure | 1.4 MPa | |
| Total Exposure Time | 15 minutes | 15 to 60 minutes |
| Pulse Duration | | 0.1 to 100 ms |

In particular, it is noted that ultrasound between 1 MHz and 3 MHz with a time average acoustic energy of approximately 1 to 2 W cm⁻² has been shown to induce a substantial enhancement of permeability and/or therapeutic enhancement as described above.

Using the techniques and apparatuses described above, one of skill in the art may determine the appropriate characteristics of the membrane to achieve the desired flow of therapeutic compound through the catheter.

The embodiments described herein facilitate radial and axial delivery of a therapeutic compound from a catheter in a substantially uniform distribution pattern. In conventional therapeutic compound delivery catheters, wherein a therapeutic compound is delivered through a ports of holes in the catheter, such radially and axially uniform delivery of medicament cannot be obtained. Additionally, the embodiments described herein will result in drug release along a significantly larger surface area than a conventional catheter having fluid delivery ports.

Furthermore, certain embodiments described herein reduce or eliminate delivery of therapeutic compound to regions where ultrasonic energy is not being applied (that is, non-clot regions). More specifically, when application of ultrasonic energy to a particular region is terminated, either because the treatment has completed, or because there is no clot in the vicinity, the delivery of therapeutic compound will also end. This configuration (1) prevents unnecessary delivery of therapeutic compound, which is advantageous if the therapeutic compound being delivered has negative secondary effects, (2) promotes efficient use of therapeutic compounds, and (3) reduces or eliminates the need to know which locations along the length of the catheter require therapy.

### Scope of the Invention

While the foregoing detailed description discloses several embodiments of the present invention, it should be understood that this disclosure is illustrative only and is not limiting of the present invention. It should be appreciated that the specific configurations and operations disclosed can differ from those described above, and that the methods described herein can be used in contexts other than treatment of an occluded vasculature.

1. A catheter configured to be positioned within a patient's vasculature, the catheter comprising:
a fluid delivery lumen;
an ultrasound radiating member positioned adjacent to at least a portion of the fluid delivery lumen; and
a semi-permeable sheath covering at least a portion of the fluid delivery lumen, such that a fluid passing from the fluid delivery lumen to the patient's vasculature crosses the sheath, wherein the sheath has an increased porosity when exposed to ultrasonic energy.

2. The catheter of claim 1, further comprising a plurality of ultrasound radiating members, wherein the plurality of ultrasound radiating members are electrically coupled into a plurality of electrical groups, and wherein each group of ultrasound radiating members is independently drivable by a control system.

3. The catheter of claim 1, further comprising a utility lumen, wherein a plurality of ultrasound radiating members are positioned on an inner core that is slidable within the utility lumen.

4. The catheter of claim 1, further comprising a temperature sensor positioned adjacent the ultrasound radiating member.

5. The catheter of claim 1, wherein the semi-permeable membrane has an average pore size between approximately 10 nm and approximately 10 μm.

6. The catheter of claim 1, wherein the semi-permeable membrane comprises a polycarbonate membrane.

7. The catheter of claim 1, further comprising a plurality of separate fluid delivery lumens, such that more than one fluid delivery lumen is at least partially covered by the semi-permeable sheath.

8. A catheter system for delivering ultrasonic energy and a therapeutic compound to a treatment site within a patient's vasculature, the catheter comprising:
a tubular body having an energy delivery section;
a fluid delivery lumen extending at least partially through the tubular body;
a semi-permeable membrane positioned along a portion of the fluid delivery lumen, the membrane having an increased porosity when exposed to ultrasonic energy; and
an inner core configured for insertion into the tubular body, the inner core comprising:
an elongate electrical conductor having a plurality of flattened regions, each flattened region having a first flat side and a second flat side opposite the first flat side, and
a plurality of ultrasound radiating members mounted in pairs to the flattened regions of the elongate electrical conductor, such that a first ultrasound radiating member is mounted to the first flat side of the elongate electrical conductor, and a second ultrasound radiating member is mounted to the second flat side of the elongate electrical conductor; and
wiring such that a voltage can be applied from the elongate electrical conductor across the first and second ultrasound radiating members, thereby allowing the first and second ultrasound radiating members to be driven simultaneously.

9. The catheter system of claim 8, wherein the ultrasound radiating members are ultrasonic transducers in the shape of a rectangular bar.

10. The catheter system of claim 8 further comprising a temperature sensor positioned adjacent the ultrasound radiating member.

11. The catheter system of claim 8, wherein the semi-permeable membrane comprises a polycarbonate membrane.

12. The catheter system of claim 8, wherein the semi-permeable membrane has an average pore size between approximately 10 nm and approximately 10 μm.

13. The catheter system of claim 8, further comprising a plurality of fluid delivery lumens.

14. The catheter system of claim 8, wherein the fluid delivery lumen includes at least one outlet in the energy delivery section, and wherein the semi-permeable membrane is positioned over the outlet, such that a fluid passing from the fluid delivery lumen to the patient's vasculature crosses the semi-permeable membrane.

15. The catheter system of claim 8, wherein at least a portion of the fluid delivery lumen is made from the semi-permeable membrane.

16. A catheter comprising:
an elongate outer sheath with an exterior surface, wherein a distal end portion of the outer sheath has a diameter of less than about 5 French, the outer sheath defining a central lumen extending longitudinally therethrough;
an elongate inner core extending through the central lumen of the outer sheath and ending at an exit port located at a catheter distal tip, the inner core defining a delivery lumen adapted for delivery of a therapeutic compound through the delivery lumen an out the exit port to a treatment site;
a cylindrical ultrasound radiating member coupled along the distal end portion of the inner core and located distal to the outer sheath; and
a semi-permeable membrane covering the exit port, such that a fluid passing from the delivery lumen to the treatment site crosses the semi-permeable membrane.

17. The catheter system of claim 16, wherein a region of the outer sheath that is positioned adjacent the ultrasound radiating member has an increased acoustic transparency.

18. The catheter system of claim 16, further comprising a temperature sensor positioned adjacent the ultrasound radiating member.

19. The catheter system of claim 16, wherein the semi-permeable membrane has an average pore size between approximately 10 nm and approximately 10 μm.

20. The catheter system of claim 16, wherein the semi-permeable membrane comprises a polycarbonate membrane.

21. The catheter system of claim 16, further comprising a stiffener ring circumscribing the exit port, the stiffener ring configured to prevent the exit port form increasing in diameter.

22. A method comprising:
positioning a catheter at a treatment site within a patient's vasculature, wherein the catheter includes an ultrasound radiating member and a fluid delivery lumen, and wherein an obstruction is located at the treatment site;
passing a therapeutic compound through the fluid delivery lumen;
passing a control signal to the ultrasound radiating member such that ultrasonic energy is generated at the treatment site, wherein generation of ultrasonic energy causes at least a portion of the therapeutic compound to pass from the fluid delivery lumen, through a semi-permeable membrane, and to the patient's vasculature.

23. The method of claim 22, further comprising a plurality of ultrasound radiating members, wherein the plurality of ultrasound radiating members are electrically coupled into a plurality of electrical groups, and wherein each group of ultrasound radiating members is independently drivable by a control system, such that a region of fluid delivery can be electrically controlled by the control system.

24. The method of claim 22, further comprising moving the ultrasound radiating member with respect to the fluid delivery lumen during delivery of ultrasonic energy, such that a region of fluid delivery moves correspondingly with movement of the ultrasound radiating member.

25. The method of claim 22, wherein the semi-permeable membrane comprises a polycarbonate membrane.

26. The method of claim 22, wherein the semi-permeable membrane has an average pore size between approximately 10 nm and approximately 10 μm.

27. The method of claim 22, further comprising:
monitoring a temperature at the treatment site with a temperature sensor positioned on the catheter; and
adjusting the control signal based at least partially on the temperature at the treatment site.
